(19) United States
(12) Patent Application Publication
(10) Pub. No.: US 2010/0179161 A1
(43) Pub. Date: Jul. 15, 2010

(54) PYRAZOLINE DERIVATIVES FOR THE TREATMENT OF TURBERCULOSIS

(75) Inventors: Balachandra Shankar Bandodkar, Bangalore (IN); Stefan Schmitt, Molndal (SE)

Correspondence Address: ASTRAZENECA R&D BOSTON, 35 GATEHOUSE DRIVE, WALTHAM, MA, 02451-1215, US

(73) Assignee: ASTRAZENECA AB, Södertälje (SE)

(21) Appl. No.: 12/063,740

(22) PCT Filed: Aug. 16, 2006
(86) PCT No.: PCT/GB06/03042

§ 371 Date: Feb. 13, 2008

(30) Foreign Application Priority Data
Aug. 19, 2005 (IN) 2222/DEL/2005

(51) Int. Cl. A61K 31/4439; C07D 401/04; C07D 401/14; A61K 31/4709; C07D 409/14; C07D 403/04; A61K 31/501; C07D 405/14; A61P 31/06

(52) U.S. Cl. 514/252.05; 546/276.1; 514/341; 546/166; 514/314; 544/238

(57) ABSTRACT

Compounds of the formula (I) and pharmaceutically acceptable salts or in vivo hydrolysable esters thereof, useful in the treatment of Mycobacterium tuberculosis (M.tb).

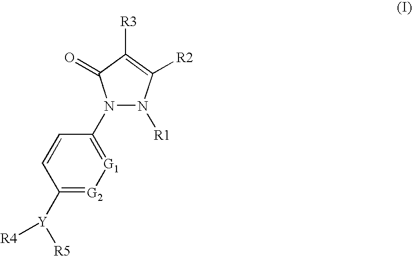

The present invention relates to chemical compounds, to their production as well as to pharmaceutical compositions containing them as well as to their use in therapy, in particular of tuberculosis.

Tuberculosis is the single largest infectious disease killer in the world that kills about 2 million people every year. Someone in the world is infected with TB every second and nearly 1% of the world population is newly infected with TB every year. Overall one third of the world's population is infected with the TB bacillus and 5 to 10% of people who are infected with TB become sick or infectious at some time during their lifetime. Drugs in use today were discovered more than 40 years ago and since then there has been no major pharmaceutical research effort to discover and develop any new therapeutic agent. There is an urgent medical need to combat this disease with drugs that will be rapidly effective against drug-resistant as well as sensitive TB.

Combination therapy for TB includes four drugs, rifampicin, isoniazid, pyrizinamide and ethambutol, given for a minimum duration of six months. Use of multiple drugs helps in preventing the appearance of drug-resistant mutants and six months of treatment helps in preventing relapse. On the other hand, multiple drug therapy and the prolonged duration of therapy are major impediments to compliance. Control programmes aimed at implementing “compliance” through DOTS (Directly Observed Therapy Service) exert a huge administrative burden on any treatment. At present, DOTS is available to only 25% of TB patients. WHO estimates that even a reduction to a 4-month therapy would allow DOTS to reach more than 50% of the TB patients world wide and thus have a direct advantage in TB control programmes. Among the four anti TB drugs, rifampicin plays a major role in shortening the duration of therapy to six months and the duration increases to 18 months in case of rifampicin resistant TB.

Mycobacterium tuberculosis shikimate kinase (MtSK) is essential for growth of Mycobacterium tuberculosis (T. Parish et al, Microbiology, 2002, 148, 3069-3077). MtSK is therefore a potential target for drug discovery purposes.

We have now discovered that certain pyrazolone derivatives are useful as inhibitors of the MtSK enzyme.

Therefore according to the present invention we provide a compound of the formula (I)

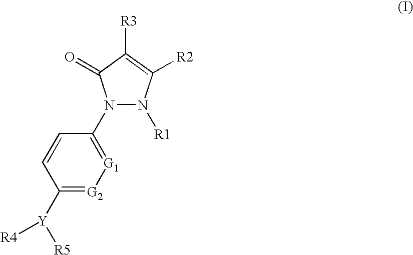

wherein G₁ and G₂ are independently selected from C or N and the aromatic ring comprising them is further optionally substituted by one or two C₁₋₆ alkyl groups;

### Y is O, N or C═O;

R1 is H or C₁₋₆ alkyl;
R2 is H or C₁₋₆ alkyl; C₆₋₁₀ aryl-C₁₋₆ alkyl-, C₆₋₁₀ heteroaryl-C₁₋₆ alkyl-, C₁₋₆ alkoxy, C₆₋₁₀ aryl-C₁₋₆ alkoxy-, C₆₋₁₀ heteroaryl-C₁₋₆ alkoxy-, or —N substituted by one or two C₁₋₄ alkyl groups;
R3 is H, C₁₋₆ alkyl, C₅₋₁₀ aryl-C₁₋₆ alkyl-, or C₆₋₁₀ heteroaryl-C₁₋₆ alkyl-, C₁₋₆ alkoxy, C₆₋₁₀ aryl-C₁₋₆ alkoxy-, C₆₋₁₀ heteroaryl-C₁₋₆ alkoxy-, or —N substituted by one or two C₁₋₄ alkyl groups;
R4 is H or C₁₋₆ alkyl, except where Y is O or C═O then R4 is absent;
R5 is C₁₋₆ alkyl, C₅₋₁₀ aryl, C₅₋₁₀ heteroaryl, C₅₋₁₀ aryl-C₁₋₆ alkyl-, C₅₋₁₀ heteroaryl-C₁₋₆ alkyl, SO₂—C₅₋₁₀ aryl or SO₂—C₅₋₁₀ heteroaryl, C═O—C₅₋₁₀ aryl or C═O—C₅₋₁₀ heteroaryl; and when Y is C═O then additionally —NH—C₅₋₁₀ aryl or —NH—C₅₋₁₀ heteroaryl,
wherein heteroaryl comprises 1-3 heteroatoms independently selected from N, O, or S and wherein each aryl or heteroaryl group is optionally substituted by 1-3 groups independently selected from C₁₋₆ alkyl, C₁₋₆ alkoxy, difluoromethyl, trifluoromethyl, difluoromethoxy, trifluoromethoxy, halogen, hydroxy, NO₂, amino, di-C₁₋₄ alkylamino, phenyl or CN;
or a pharmaceutically acceptable salt or in vivo hydrolysable ester thereof.

In this specification the term ‘alkyl’ when used either alone or as a suffix includes straight chained or branched and cyclic structures. These groups contain up to 6 carbon atoms such as methyl, ethyl, propyl, isopropyl, butyl, t-butyl, and isobutyl, pentyl, hexyl and may contain one or more unsaturations and one or more chiral centres.

The term “halo” includes fluoro, chloro, bromo and iodo, such as for example fluoro, chloro and bromo; fluoro, chloro; fluoro; chloro; bromo.

References to “aryl” includes aromatic carbocylic groups of up to 10 carbon atoms, for example of up to 6 carbon atoms. Examples include naphthyl and phenyl groups.

“Heteroaryl” refers to heterocyclic groups which have an aromatic character and comprise up to 10 ring atoms. These include monocyclic or bicyclic aryl rings containing 5 to 10 ring atoms of which 1, 2, 3 or 4 ring atoms are chosen from nitrogen, sulphur and oxygen. Examples of such rings include pyrrolyl, furanyl, thienyl, thiazolyl, isothiazolyl, oxazolyl, isoxazolyl, imidazolyl, pyrazolyl, triazolyl, tetrazolyl, pyridyl, pyrimidinyl, pyrazinyl, pyridazinyl, triazinyl, benzfuranyl, benzthieno, indolyl, benzimidazolyl, benzoxazolyl, benzthiazolyl, indazolyl, benzisoxazolyl, benzisothiazolyl, benztriazolyl, quinolinyl, isoquinolinyl and naphthiridinyl.

Examples of convenient heterocyclic groups include thienyl, pyridyl, and quinolinyl.

The term “aralkyl” refers to aryl substituted alkyl groups of up to 16 carbon atoms, such as of up to 10 or 8 carbon atoms in particular phenethyl or benzyl, more particularly benzyl groups.

The term “heteroaralkyl” refers to alkyl groups of up to 6 carbon atoms linked to a heteroaryl moiety of up to 10 ring atoms.

Conveniently (taken together or each independently),

### G₁ is N;

### G₂ is C;

### Y is N; Y is O; Y is C═O;

### R1 is H;

R2 is H; C₁₋₄ alkyl such as ethyl or methyl;
R3 is H or C₁₋₄ alkyl, aralkyl of up to 12 carbon atoms such as phenethyl or benzyl;

### R4 is H;

R5 is SO₂—C₅₋₁₀ aryl or SO₂—C₅₋₁₀ heteroaryl, each optionally substituted by up to 3 substituents independently selected from C₁₋₄ alkyl, C₁₋₄ alkoxy, difluoromethyl, trifluoromethyl, difluoromethoxy, trifluoromethoxy, halogen, hydroxy or NO₂.

More conveniently (taken together or each independently),

### G₁ is N;

### G₂ is C;

### Y is N or C═O;

### R1 is H;

R2 is ethyl or methyl, in particular methyl;
R3 is ethyl or methyl, in particular aralkyl of up to 10 carbon atoms such as phenethyl or benzyl;

### R4 is H;

R5 is SO₂— phenyl, SO₂— naphthyl, or SO₂— thienyl, each optionally substituted by up to 3 substituents independently selected from methyl, ethyl, propyl, i-propyl, i-butyl, methoxy, di-fluoromethyl, difluoromethoxy, chlorine, fluorine, bromine, hydroxy or NO₂.

Particular compounds of the invention (taken together or each independently) are:
Benzenesulfonamide, N-[6-(2,5-dihydro-3-methyl-5-oxo-1H-pyrazol-1-yl)-3-pyridinyl]-3-methoxy.
Benzenesulfonamide, N-[6-(2,5-dihydro-3-methyl-5-oxo-1H-pyrazol-1-yl)-3-pyridinyl]-4-methoxy.
Benzenesulfonamide, N-[6-(2,5-dihydro-3-methyl-5-oxo-1H-pyrazol-1-yl)-3-pyridinyl]-4-(trifluoromethoxy).
Benzenesulfonamide, N-[6-(2,5-dihydro-3-methyl-5-oxo-1H-pyrazol-1-yl)-3-pyridinyl].
Benzenesulfonamide, N-[6-(2,5-dihydro-3-methyl-5-oxo-1H-pyrazol-1-yl)-3-pyridinyl]-3-fluoro.
Benzenesulfonamide, 3-bromo-N-[6-(2,5-dihydro-3-methyl-5-oxo-1H-pyrazol-1-yl)-3-pyridinyl].
Benzenesulfonamide, 3-chloro-N-[6-(2,5-dihydro-3-methyl-5-oxo-1H-pyrazol-1-yl)-3-pyridinyl]-4-fluoro.
Benzenesulfonamide, N-[6-(2,5-dihydro-3-methyl-5-oxo-1H-pyrazol-1-yl)-3-pyridinyl]-3-nitro.
Benzenesulfonamide, N-[6-(2,5-dihydro-3-methyl-5-oxo-1H-pyrazol-1-yl)-3-pyridinyl]-4-propyl.
Benzenesulfonamide, N-[6-(2,5-dihydro-3-methyl-5-oxo-1H-pyrazol-1-yl)-3-pyridinyl]-2,3,4-trifluoro.
Benzenesulfonamide, N-[6-(2,5-dihydro-3-methyl-5-oxo-1H-pyrazol-1-yl)-3-pyridinyl]-3-methyl.
Benzenesulfonamide, 3-chloro-N-[6-(2,5-dihydro-3-methyl-5-oxo-1H-pyrazol-1-yl)-3-pyridinyl].
Benzenesulfonamide, N-[6-(2,5-dihydro-3-methyl-5-oxo-1H-pyrazol-1-yl)-3-pyridinyl]-2-methyl.
Benzenesulfonamide, N-[6-(2,5-dihydro-3-methyl-5-oxo-1H-pyrazol-1-yl)-3-pyridinyl]-4-(1-methylethyl).
Benzenesulfonamide, 4-(difluoromethoxy)-N-[6-(2,5-dihydro-3-methyl-5-oxo-1H-pyrazol-1-yl)-3-pyridinyl].
Benzenesulfonamide, 3-(difluoromethoxy)-N-[6-(2,5-dihydro-3-methyl-5-oxo-1H-pyrazol-1-yl)-3-pyridinyl].
Benzenesulfonamide, 4-chloro-N-[6-(2,5-dihydro-3-methyl-5-oxo-1H-pyrazol-1-yl)-3-pyridinyl].
Benzenesulfonamide, N-[6-(2,5-dihydro-3-methyl-5-oxo-1H-pyrazol-1-yl)-3-pyridinyl]-3-(trifluoromethyl).
Benzenesulfonamide, N-[6-(2,5-dihydro-3-methyl-5-oxo-1H-pyrazol-1-yl)-3-pyridinyl]-4-methoxy-2-nitro.
2-Naphthalenesulfonamide, N-[6-(2,5-dihydro-3-methyl-5-oxo-1H-pyrazol-1-yl)-3-pyridinyl].
1-Naphthalenesulfonamide, N-[6-(2,5-dihydro-3-methyl-5-oxo-1H-pyrazol-1-yl)-3-pyridinyl].
Benzenesulfonamide, N-[6-(2,5-dihydro-3-methyl-5-oxo-1H-pyrazol-1-yl)-3-pyridinyl]-3,5-dimethyl.
Benzenesulfonamide, N-[6-(2,5-dihydro-3-methyl-5-oxo-1H-pyrazol-1-yl)-3-pyridinyl]-3,5-dimethyl.
Benzenesulfonamide, N-[6-(2,5-dihydro-3-methyl-5-oxo-1H-pyrazol-1-yl)-3-pyridinyl]-3-(trifluoromethoxy).
Benzenesulfonamide, 4-bromo-N-[6-(2,5-dihydro-3-methyl-5-oxo-1H-pyrazol-1-yl)-3-pyridinyl].
Benzenesulfonamide, N-[6-(2,5-dihydro-3-methyl-5-oxo-1H-pyrazol-1-yl)-3-pyridinyl]-2,4-difluoro.
Benzenesulfonamide, N-[6-(2,5-dihydro-3-methyl-5-oxo-1H-pyrazol-1-yl)-3-pyridinyl]-4-(1,1-dimethylethyl).
8-Quinolinesulfonamide, N-[6-(2,5-dihydro-3-methyl-5-oxo-1H-pyrazol-1-yl)-3-pyridinyl].
Benzenesulfonamide,3,4-dichloro-N-[6-(2,5-dihydro-3-methyl-5-oxo-1H-pyrazol-1-yl)-3-pyridinyl].
3-Thiophenesulfonamide, 2,5-dichloro-N-[6-(2,5-dihydro-3-methyl-5-oxo-1H-pyrazol-1-yl)-3-pyridinyl]
Benzenesulfonamide,N-[6-(2,5-dihydro-3-methyl-5-oxo-1H-pyrazol-1-yl)-3-pyridinyl]-3,5-difluoro.
Benzenesulfonamide,3,5-dichloro-N-[6-(2,5-dihydro-3-methyl-5-oxo-1H-pyrazol-1-yl)-3-pyridinyl].
Benzenemethanesulfonamide,N-[6-(2,5-dihydro-3-methyl-5-oxo-1H-pyrazol-1-yl)-3-pyridinyl].
Benzenesulfonamide,3,5-dichloro-N-[6-(2,5-dihydro-3-methyl-5-oxo-1H-pyrazol-1-yl)-3-pyridinyl]-2-hydroxy.
Benzenesulfonamide,2-bromo-N-[6-(2,5-dihydro-3-methyl-5-oxo-1H-pyrazol-1-yl)-3-pyridinyl]
Benzenesulfonamide,2,4-dichloro-N-[6-(2,5-dihydro-3-methyl-5-oxo-1H-pyrazol-1-yl)-3-pyridinyl].
Benzenesulfonamide,5-bromo-N-[6-(2,5-dihydro-3-methyl-5-oxo-1H-pyrazol-1-yl)-3-pyridinyl]-2-methoxy.
Benzenesulfonamide,N-[6-(2,5-dihydro-3-methyl-5-oxo-1H-pyrazol-1-yl)-3-pyridinyl]-3,4-dimethyl.
Benzenesulfonamide,N-[6-(2,5-dihydro-3-methyl-5-oxo-1H-pyrazol-1-yl)-3-pyridinyl]-2,5-dimethoxy.
N-[6-(4-Benzyl-3-methyl-5-oxo-2,5-dihydro-1H-pyrazol-1-yl)pyridin-3-yl]-3-nitrobenzenesulfonamide.
N-[6-(4-Benzyl-3-methyl-5-oxo-2,5-dihydro-1H-pyrazol-1-yl)pyridin-3-yl]-4-fluorobenzenesulfonamide.
Benzenesulfonamide,N-[6-[2,5-dihydro-3-methyl-5-oxo-4-(phenylmethyl)-1H-pyrazol-1-yl]-3-pyridinyl]-4-propyl.
Benzenesulfonamide,3-chloro-N-[6-[2,5-dihydro-3-methyl-5-oxo-4-(phenylmethyl)1H-pyrazol-1-yl]-3-pyridinyl].
Benzenesulfonamide,N-[6-[2,5-dihydro-3-methyl-5-oxo-4-(phenylmethyl)-1H-pyrazol-1-yl]-3-pyridinyl]-4-(1,1-dimethylethyl).
1-Naphthalenesulfonamide,N-[6-[2,5-dihydro-3-methyl-5-oxo-4-(phenylmethyl)-1H-pyrazol-1-yl]-3-pyridinyl].
Benzenesulfonamide,3-chloro-N-[6-[2,5-dihydro-3-methyl-5-oxo-4-(phenylmethyl)-1H-pyrazol-1-yl]-3-pyridinyl]-4-fluoro.
3-fluoro-N-[6-(3-methyl-5-oxo-2,5-dihydro-1H-pyrazol-1-yl)pyridin-3-yl]benzamide.
4-tert-butyl-N-[6-(3-methyl-5-oxo-2,5-dihydro-1H-pyrazol-1-yl)pyridin-3-yl]benzamide.
4-fluoro-N-[6-(3-methyl-5-oxo-2,5-dihydro-1H-pyrazol-1-yl)pyridin-3-yl]benzamide.
4-cyano-N-[6-(3-methyl-5-oxo-2,5-dihydro-1H-pyrazol-1-yl)pyridin-3-yl]benzamide.
3-cyano-N-[6-(3-methyl-5-oxo-2,5-dihydro-1H-pyrazol-1-yl)pyridin-3-yl]benzamide.
N-[6-(3-methyl-5-oxo-2,5-dihydro-1H-pyrazol-1-yl)pyridin-3-yl]-4-(trifluoromethyl)benzamide.
N-[6-(3-methyl-5-oxo-2,5-dihydro-1H-pyrazol-1-yl)pyridin-3-yl]-4-(trifluoromethoxy)benzamide.
4-(dimethylamino)-N-[6-(3-methyl-5-oxo-2,5-dihydro-1H-pyrazol-1-yl)pyridin-3-yl]benzamide.
2-methoxy-N-[6-(3-methyl-5-oxo-2,5-dihydro-1H-pyrazol-1-yl)pyridin-3-yl]benzamide.
4-methyl-N-[6-(3-methyl-5-oxo-2,5-dihydro-1H-pyrazol-1-yl)pyridin-3-yl]benzamide.
N-[6-(3-methyl-5-oxo-2,5-dihydro-1H-pyrazol-1-yl)pyridin-3-yl]thiophene-2-carboxamide.
2-fluoro-N-[6-(3-methyl-5-oxo-2,5-dihydro-1H-pyrazol-1-yl)pyridin-3-yl]benzamide.
3-(dimethylamino)-N-[6-(3-methyl-5-oxo-2,5-dihydro-1H-pyrazol-1-yl)pyridin-3-yl]benzamide.
N-[6-(3-methyl-5-oxo-2,5-dihydro-1H-pyrazol-1-yl)pyridin-3-yl]benzamide.
Benzamide, 3-cyano-N-[6-[2,5-dihydro-3-methyl-5-oxo-4-(phenylmethyl)-1H-pyrazol-1-yl]-3-pyridinyl].
Benzamide, 4-cyano-N-[6-[2,5-dihydro-3-methyl-5-oxo-4-(phenylmethyl)-1H-pyrazol-1-yl]-3-pyridinyl].
2-{6-[2-(4-aminophenyl)ethoxy]pyridazin-3-yl}-5-methyl-1,2-dihydro-3H-pyrazol-3-one.
2-[6-(1,3-benzodioxol-5-ylmethoxy)pyridazin-3-yl]-5-methyl-1,2-dihydro-3H-pyrazol-3-one.
2-{6-[(4-methoxybenzyl)oxy]pyridazin-3-yl}-5-methyl-1,2-dihydro-3H-pyrazol-3-one.
5-methyl-2-(6-{[4-(trifluoromethoxy)benzyl]oxy}pyridazin-3-yl)-1,2-dihydro-3H-pyrazol-3-one.
2-{6-[(3-aminobenzyl)oxy]pyridazin-3-yl}-5-methyl-1,2-dihydro-3H-pyrazol-3-one.
5-methyl-2-(6-{[4-(trifluoromethyl)benzyl]oxy}pyridazin-3-yl)-1,2-dihydro-3H-pyrazol-3-one.
5-methyl-2-(6-{[3-(trifluoromethyl)benzyl]oxy}pyridazin-3-yl)-1,2-dihydro-3H-pyrazol-3-one.
2-(6-[(4-fluorobenzyl)oxy]pyridazin-3-yl)-5-methyl-1,2-dihydro-3H-pyrazol-3-one.
2-[6-(benzyloxy)pyridazin-3-yl]-5-methyl-1,2-dihydro-3H-pyrazol-3-one.
2-[6-(1,1′-biphenyl-4-ylmethoxy)pyridazin-3-yl]-5-methyl-1,2-dihydro-3H-pyrazol-3-one.
5-methyl-2-{6-[(4-methylbenzyl)oxy]pyridazin-3-yl}-1,2-dihydro-3H-pyrazol-3-one.
2-{6-[(2,4-dichlorobenzyl)oxy]pyridazin-3-yl}-5-methyl-1,2-dihydro-3H-pyrazol-3-one.
2-{6-[(2,5-dimethylbenzyl)oxy]pyridazin-3-yl}-5-methyl-1,2-dihydro-3H-pyrazol-3-one.
5-methyl-2-{6-[(3-methylbenzyl)oxy]pyridazin-3-yl}-1,2-dihydro-3H-pyrazol-3-one.
2-{6-[(3-chlorobenzyl)oxy]pyridazin-3-yl}-5-methyl-1,2-dihydro-3H-pyrazol-3-one.
2-[6-(2-furylmethoxy)pyridazin-3-yl]-5-methyl-1,2-dihydro-3H-pyrazol-3-one.

Suitable pharmaceutically acceptable salts of compounds of formula (I) include acid addition salts such as methanesulfonate, fumarate, hydrochloride, hydrobromide, citrate, maleate and salts formed with phosphoric and sulphuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. There may be more than one cation or anion depending on the number of charged functions and the valency of the cations or anions. A preferred pharmaceutically acceptable salt is a sodium salt.

An in vivo hydrolysable ester of a compound of the formula (I) containing carboxy or hydroxy group is, for example, a pharmaceutically acceptable ester which is hydrolysed in the human or animal body to produce the parent acid or alcohol.

Suitable pharmaceutically acceptable esters for carboxy include alkyl esters, such as C₁₋₆ alkyl esters for example, ethyl esters, C₁₋₆alkoxymethyl esters for example methoxymethyl, C₁₋₆alkanoyloxymethyl esters for example pivaloyloxymethyl, phthalidyl esters, C₃₋₃cycloalkoxy-carbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

Suitable pharmaceutically acceptable esters of compounds of formula (I) are in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

Esters which are not in vivo hydrolysable are useful as intermediates in the production of the compounds of formula (I) and therefore these form a further aspect of the invention.

Compounds of formula (I) are suitably prepared as follows:

(i) where Y is N,
by reacting a compound of formula (II)

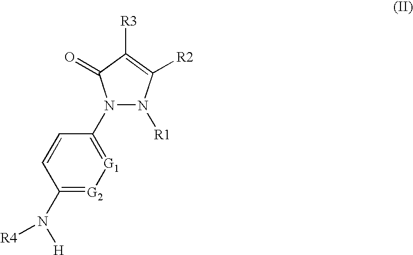

wherein R¹, R², R³, R⁴, G₁ and G₂ are as defined in relation to formula (I), with a compound of formula (III)

R5-SO₂—Z (III)

wherein R⁵ is as defined in relation to formula (I), and
wherein Z is a leaving group (such as chloro, bromo, iodo, O-alkyl, O-aryl, O-heteroaryl), under appropriate reaction conditions;
(ii) where Y is N, by reacting a compound of formula II as defined above, with a compound of formula (IV)

R⁵—CO—Z (IV)

wherein R⁵ is as defined in relation to formula (I), and
wherein Z is a leaving group (such as hydroxy or Cl), under appropriate reaction conditions;
(iii) Y is O, by reacting a compound of formula (V)

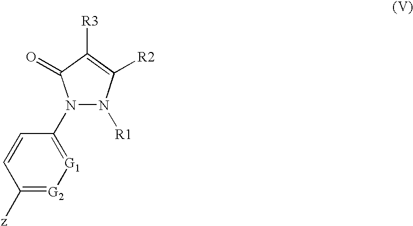

wherein R¹, R², R³, G₁ and G₂ are as defined in relation to formula (I),
wherein Z is a leaving group (such as chloro, bromo, iodo, O-alkyl, O-aryl, O-heteroaryl), with a compound of the formula (VI)

R⁵—OH (VI)

wherein R⁵ is as defined in relation to formula (I)
and thereafter if desired or necessary converting any substituent group to another substituent group as defined.

Any convenient leaving group Z may be used. Examples of such groups are provided in standard chemistry textbooks such as “Organic Chemistry” by Jonathan Clayden et al, published by Oxford University Press (3^{rd} Edn 2005). They include hydroxy and halogen such as chloro or bromo.

Compounds of formula (I) are suitably prepared as follows:
(i) Where Y is N, reaction of compounds of formula (II) wherein R¹, R², R³, R⁴, G₁ and G₂ are as defined in relation to formula (I), with sulfonyl chloride (R⁵SO₂Cl), where R⁵ is as defined in formula (I), can be carried out in the presence of a suitable base and solvent at temperature ranging from 0° C. to room temperature. Examples of suitable bases include pyridine, triethylamine, diisopropyl ethyl amine. In particular pyridine is used. Suitable solvents include chlorinated solvents such as chloroform and dichloromethane, or ethers such as tetrahydrofuran, 1,4-dioxane. In particular dichloromethane is used. The temperature of the reaction can be performed between 0° C. and room temperature, preferably at 0° C.
(ii) Where Y is N, reaction of compounds of formula (II) wherein R¹, R², R³, R⁴, G₁ and G₂ are as defined in relation to formula (I), with acid (R⁵CO₂H), where R⁵ is as defined in formula (I), can be carried out in the presence of a suitable coupling reagent and a base in a solvent at temperature ranging from 0° C. to room temperature. Examples of suitable coupling agents include dicyclohexylcarbodiimide (DCC), 1-(3-dimethylaminopropyl)-3-ethylcarbodiimide hydrochloride (EDCI) and 2-(7-Aza-1h-benzotriazole-1-yl)-1,1,3,3-tetramethyluronium hexafluorophosphate (HATU). Most preferably EDCI is used. Bases include pyridine, triethylamine, diisopropyl ethyl amine and 4-Dimethylaminopyridine (DMAP). Most preferably DMAP is used. Suitable solvents include chlorinated solvents such as chloroform and dichloromethane, or ethers such as tetrahydrofuran, 1,4-dioxane. Preferably dichloromethane is used. The temperature of the reaction can be performed between 0° C. and room temperature, preferably at room temperature.
(iii) Y is O, by reacting a compound of formula (V) wherein R¹, R², R³, G₁ and G₂ are as defined in relation to formula (I) with R⁵OH, wherein R⁵ is as defined in relation to formula (I), can be carried out in the presence of a suitable base in a solvent at temperature ranging from room temperature to reflux temperature. Examples of suitable bases include metal alkoxides such as those from caesium, potassium, lithium or sodium. Most preferably potassium tert-butoxide is used. Suitable solvents include ethers such as tetrahydrofuran, 1,4-dioxane, glyme and diglyme. Preferably tetrahydrofuran is used. The temperature of the reaction can be performed between 10° C. and 120° C., preferably at 70° C.

Compounds of formula (II) etc. are either known compounds or they may be prepared from known compounds by conventional literature methods.

According to a further aspect of the invention there is provided a compound of the formula (I) as defined herein, or a pharmaceutically acceptable salt or an in vivo hydrolysable ester thereof; for use in a method of treatment of the human or animal body by therapy. In particular, the compounds are used in methods of treatment of M.tb.

According to a further aspect of the present invention there is provided a treatment method for M.Tb by inhibiting MtSK, which comprises administering to said human or animal an effective amount of a compound of formula (I), or a pharmaceutically acceptable salt, or an in vivo hydrolysable ester thereof.

The invention also provides a pharmaceutical composition comprising a compound of formula (I) as defined herein, or a pharmaceutically acceptable salt, or an in vivo hydrolysable ester thereof, in combination with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients that may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the Formula I will naturally vary according to the nature and severity of the conditions, the age and sex of the animal or patient and the route of administration, according to well known principles of medicine. As mentioned above, compounds of the Formula I are useful in treating diseases or medical conditions which are due alone or in part to the effects of farnesylation of rats.

In using a compound of the Formula I for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

### Materials and Methods:

### Protein Purification

Mycobacterium tuberculosis Shikimate Kinase (MtSK) protein was prepared according to the protocol set out in J. S. Oliveira et al, Protein Expression and Purification, 2001, 22, 430-435.

Gene coding for Mycobacterium tuberculosis shikimate kinase (MtSK)-aroK, Rv 2539C) was cloned in pET15b plasmid so that the histidine tag was introduced at the N-terminus followed by a thrombin cleavage site (20 amino acid N-terminal tag). E. coli BL21(DE3) cells transformed with this plasmid were grown in Luria broth at 37° C. till the OD₆₀₀ nm reached 0.6. Expression of MtSK was induced by adding 1 mM IPTG followed by overnight incubation at 20° C. Cells were lysed by sonication and the His-tagged Mtsk present in the cytosolic fraction was purified using metal ion affinity column (Ni-Nitriloacetic acid(NTA) obtained from QIAGEN). The purified protein was treated with thrombin and re-purified using the affinity column. The protein was 95% pure after re-purification

### Enzyme Assay

Activity of Mycobacterium tuberculosis shikimate kinase (MtSK) was measured in a coupled assay format wherein ADP formed after the formation of shikimate phosphate through hydrolysis of ATP was detected using pyruvate kinase (PK) and lactate dehydrogenase (LDH). Oxidation of NADH to NAD during PK-LDH activity was monitored at 340 nm. Assay mixture contained 100 mM Tris.Cl, pH 7.5, 100 mM NaCl, 5 mM MgCl₂, 0.001% w/v Brij 35, 0.2 mM ATP, 0.4 mM Shikimic acid, 1 mM phosphoenolpyruvate, 0.15 mM NADH, 2 U/ml of PK-LDH and 200 ng/ml of MtSK protein in 100 microliters. Assay was performed at room temperature in 96 well half area microtitre plates (Corning Inc.) and OD₃₄₀ nm was measured using Spectramax (Molecular Devices Inc.) spectrophotometer. Initial reading was taken at 0 minutes and the final reading at the end of 60 minutes. The difference between the initial and final OD₃₄₀ nm was used to calculate activity.

When tested in the above enzyme assay all the exemplified compounds have an IC₅₀ of less than 20 μM.

The invention will now be illustrated but not limited by reference to the following Examples.

### EXAMPLE 1

### N-[6-(2,5-dihydro-3-methyl-5-oxo-1H-pyrazol-1-yl)-3-pyridinyl]-3-methoxybenzenesulfonamide

### Step A: 2-hydrazino-5-nitropyridine hydrochloride

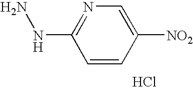

In a 250 mL round bottom flask, hydrazine hydrate (3.15 g, 3.07 mL, 63.07 mmol) was added to the suspension of 2-chloro-5-nitropyridine (5 g, 31.53 mmol). The suspension turned into green colored solution. Within a few minutes a green colored precipitate started appearing. The mixture was stirred for 2 h at room temperature. The solid was filtered at pump in vacuo, washed with ethanol and dried in vacuo to afford the title compound as the bright green colored solid (5.5 g, 91%).

MS (ES⁺): 154; ¹H NMR (DMSO-d₆, ppm): δ 4.70 (br s, 3H), 6.80 (br s, 1H), 8.18 (s, 1H), 8.88 (s, 1H), 9.23 (s, 1H).

### Step B: 1,2-dihydro-5-methyl-2-(5-nitro-2-pyridinyl)-3H-pyrazol-3-one

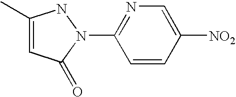

In a 80 mL CEM microwave reactor tube, ethyl acetoacetate (4.56 g, 4.4 mL, 35.03 mmol) was added to the suspension of 2-hydrazino-5-nitropyridine hydrochloride (4.5 g, 23.61 mmol) in ethanol (25 mL). The mixture was stirred at RT for 15 minutes and then microwaved (150 W) at 150° C. for 45 minutes. A yellow crystalline precipitate was observed in the reaction mixture. It was then cooled in ice-bath, crystals were filtered, washed with cold ethanol and dried in vacuo to afford the title compound as a yellow crystalline solid (3.8 g, 73%).

MS (ES⁺): 220.1; ¹H NMR. (DMSO-d₅, δ ppm): δ 2.20 (s, 3H), 5.19 (s, 1H), 8.68 (s, 2H), 9.21 (s, 1H), 12.38 (br s, 1H).

### Step C: 2-(5-amino-2-pyridinyl)-1,2-dihydro-5-methyl-3H-pyrazol-3-one

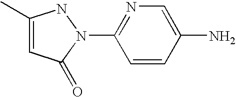

The suspension of the intermediate from step B (3.0 g, 13.64 mmol) in methanol (30 mL) containing glacial acetic acid (3 mL) and 10% Pd—C (0.5 g) was hydrogenated under 40 psi of H₂ for 2.5 h. The reaction mixture was filtered through Celite® bed to remove Pd—C. Celite® bed was thoroughly washed with methanol containing 5% acetic acid. Filtrates were combined and solvent was evaporated under vacuum. The residual syrupy mass was suspended in ethyl acetate (20 mL) and diluted with hexane (100 mL). A suspension of crystalline yellow colored solid was obtained. It was stirred for 10 min and the solid was filtered, washed with hexane and dried in vacuo to afford desired compound as greenish yellow colored crystalline solid (2.3 g, 89%).

MS (ES⁺): 190.1; ¹H NMR (DMSO-d₆, ppm): δ 2.12 (s, 3H), 5.21 (s, 1H), 5.38 (br s, 2H), 7.15 (dd, 1H), 7.71 (s, 1H), 7.75 (d, 1H), 12.00 (br s, 1H).

### Step D: N-[6-(2,5-dihydro-3-methyl-5-oxo-1H-pyrazol-1-yl)-3-pyridinyl]-3-methoxybenzenesulfonamide

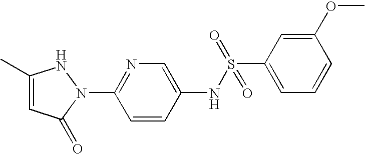

In a 10 mL reactor tube, pyridine (1 mL) was added to the solution of 2-(5-amino-2-pyridinyl)-1,2-dihydro-5-methyl-3H-pyrazol-3-one (0.19 g, 1 mmol) in 2 mL dichloromethane. The mixture was cooled to 0° C. To the cold mixture, 3-methoxybenzenesulfonyl chloride (0.21 g, 1 mmol) was added drop-wise. The reaction mixture was stirred at 0° C. for 3 h. It was then diluted with dichloromethane (20 mL) and was washed with 10% hydrochloric acid (2×10 mL), water (2×10 mL) and brine (10 mL). The extracts were dried (Na₂SO₄) and solvent was evaporated under vacuum. The residue was dissolved in methanol (5 mL). To the solution, 10% sodium hydroxide solution (2 mL) was added. The mixture was stirred overnight. It was then diluted with water (10 mL) and acidified with glacial acetic acid. Precipitated solid was filtered in vacuo, washed with cold water and dried under vacuum. The crude solid was suspended in ethyl acetate (10 mL) and sonicated for few minutes. Filtered, washed with ethyl acetate and dried in vacuo to afford the title compound as a light brown colored solid (62%).

MS (ES⁺): 361.1; ¹HNMR (DMSO-d₆, ppm): δ 2.1 (s, 3H), 3.75 (s, 3H), 5.05 (s, 1H), 7.2 (m, 3H), 7.45 (m, 1H), 7.55 (m, 1H), 8.05 (s, 1H), 8.3 (d, 1H), 10.35 (s, 1H), 11.95 (s, 1H).

The compounds set out below were prepared in the same way as in Example 1, using the appropriate starting materials.

| Ex- | | | | |
|---|---|---|---|---|
| am- | | | | MASS |
| ple | Structure | Name | NMR | (ES+) |
| 2 | 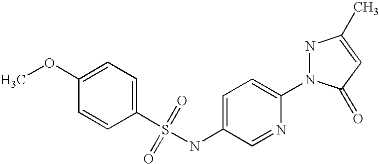 | benzenesulfonamide, N-[6- (2,5-dihydro-3-methyl-5- oxo-1H-pyrazol-1-yl)-3- pyridinyl]-4-methoxy- | 1HNMR (CDCl3, ppm): δ 2.25 (s, 3H), 3.50 (s, 1H), 3.85 (s, 3H), 5.45 (s, 1H), 6.50 (s, 1H), 6.95 (d, 2H), 7.65 (d, 2H), 7.80 (br.s, 1H), 7.95 (s, 1H) | 361 |
| 3 | 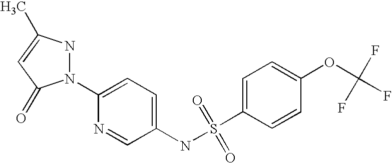 | benzenesulfonamide, N-[6- (2,5-dihydro-3-methyl-5- oxo-1H-pyrazol-1-yl)-3- pyridinyl]-4- (trifluoromethoxy)- | 1HNMR (DMSO, ppm): δ 2.1 (s, 3H), 5.1 (s, 1H), 7.5 (d, 3H), 7.85 (d, 2H), 8.1 (s, 1H), 8.35 (d, 1H), 10.5 (br.s, 1H), 11.9 (s, 1H) | 415 |
| 4 | 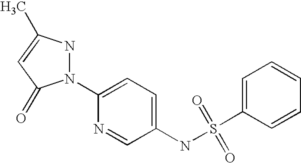 | benzenesulfonamide, N-[6- (2,5-dihydro-3-methyl-5- oxo-1H-pyrazol-1-yl)-3- pyridinyl]- | 1HNMR (DMSO, ppm): δ 2.1 (s, 3H), 5.05 (s, 1H), 7.55 (m, 4H), 7.7 (d, 2H), 8.05 (s, 1H), 8.3 (d, 1H), 10.4 (br.s, 1H), 11.9 (s, 1H) | 331 |
| 5 | 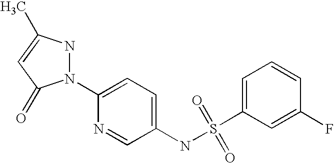 | benzenesulfonamide, N-[6- (2,5-dihydro-3-methyl-5- oxo-1H-pyrazol-1-yl)-3- pyridinyl]-3-fluoro- | 1HNMR (DMSO, ppm): δ 2.1 (s, 3H), 5.05 (br.s, 1H), 7.55 (m, 5H), 8.05 (s, 1H), 8.3 (br.s, 1H), 10.5 (br.s, 1H), 11.9 (br.s, 1H) | 349 |
| 6 | 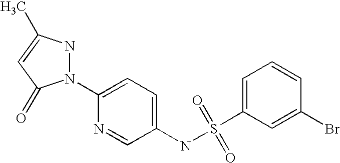 | benzenesulfonamide, 3- bromo-N-[6-(2,5-dihydro-3- methyl-5-oxo-1H-pyrazol-1- yl)-3-pyridinyl]- | 1HNMR (DMSO, ppm): δ 2.1 (s, 3H), 5.05 (s, 1H), 7.6 (m, 3H), 7.85 (d, 2H), 8.05 (s, 1H), 8.3 (d, 1H), 10.5 (br.s, 1H), 11.9 (br.s, 1H) | 411 |
| 7 | 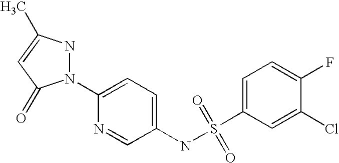 | benzenesulfonamide, 3- chloro-N-[6-(2,5-dihydro-3- methyl-5-oxo-1H-pyrazol-1- yl)-3-pyridinyl]-4-fluoro- | 1HNMR (DMSO, ppm): δ 2.1 (s, 3H), 5.05 (s, 1H), 7.6 (m, 3H), 7.9 (d, 1H), 8.05 (s, 1H), 8.3 (d, 1H), 10.5 (br.s, 1H), 11.95 (s, 1H) | 383 |
| 8 | 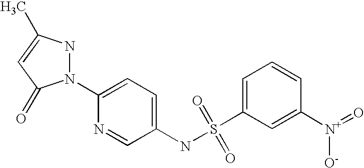 | benzenesulfonamide, N-[6- (2,5-dihydro-3-methyl-5- oxo-1H-pyrazol-1-yl)-3- pyridinyl]-3-nitro- | 1HNMR (DMSO, ppm): δ 2.1 (s, 3H), 5.05 (s, 1H), 7.6 (br.s, 1H), 7.8 (t, 1H), 8.05 (d, 2H), 8.3 (br.s, 1H), 8.45 (m, 2H) 10.7 (br.s, 1H), 11.9 (s, 1H) | 376 |
| 9 | 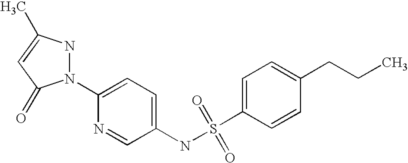 | benzenesulfonamide, N-[6- (2,5-dihydro-3-methyl-5- oxo-1H-pyrazol-1-yl)-3- pyridinyl]-4-propyl- | 1HNMR (DMSO, ppm): δ 2.15 (s, 3H), 5.15 (br.s, 1H), 7.35 (d, 2H), 7.6 (m, 3H), 8.05 (s, 2H), 10.4 (br.s, 1H), 11.9 (br.s, 1H) | 372 |
| 10 | 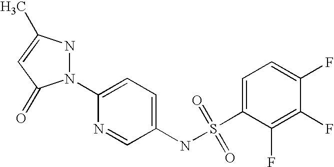 | benzenesulfonamide, N-[6- (2,5-dihydro-3-methyl-5- oxo-1H-pyrazol-1-yl)-3- pyridinyl]-2,3,4-trifluoro- | 1HNMR (DMSO, ppm): δ 2.1 (s, 3H), 5.05 (s, 1H), 7.55 (m, 3H), 8.1 (s, 1H), 8.3 (br.d, 1H), 10.9 (br.s, 1H), 11.9 (s, 1H) | 385 |
| 11 | 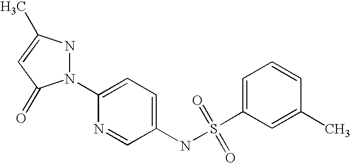 | benzenesulfonamide, N-[6- (2,5-dihydro-3-methyl-5- oxo-1H-pyrazol-1-yl)-3- pyridinyl]-3-methyl- | 1HNMR (DMSO, ppm): δ 2.1 (s, 3H), 2.35 (s, 3H), 5.05 (s, 1H), 7.5 (m, 5H), 8.05 (s, 1H), 8.3 (d, 1H), 10.3 (s, 1H), 11.9 (s, 1H) | 345 |
| 12 | 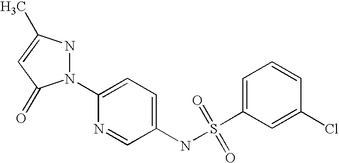 | benzenesulfonamide, 3- chloro-N-[6-(2,5-dihydro-3- methyl-5-oxo-1H-pyrazol-1- yl)-3-pyridinyl]- | 1HNMR (DMSO, ppm): δ 2.15 (s, 3H), 5.05 (s, 1H), 7.6 (m, 3H), 7.75 (d, 2H), 8.05 (s, 1H), 8.3 (d, 1H), 10.45 (s, 1H), 11.95 (s, 1H) | 365 |
| 13 | 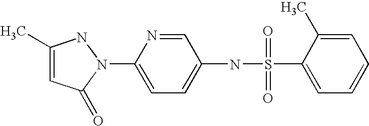 | benzenesulfonamide, N-[6- (2,5-dihydro-3-methyl-5- oxo-1H-pyrazol-1-yl)-3- pyridinyl]-2-methyl- | 1HNMR (DMSO, ppm): δ 2.1 (s, 3H), 2.6 (s, 3H), 5.05 (s, 1H), 7.3 (m, 2H), 7.5 (m, 2H), 7.85 (d, 1H), 8.05 (s, 1H), 8.25 (d, 1H), 10.45 (s, 1H), 11.9 (s, 1H) | 345 |
| 14 | 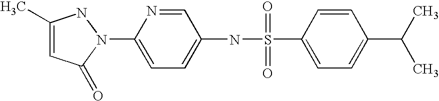 | benzenesulfonamide, N-[6- (2,5-dihydro-3-methyl-5- oxo-1H-pyrazol-1-yl)-3- pyridinyl]-4-(1- methylethyl)- | 1HNMR (DMSO, ppm): δ 1.15 (s, 6H), 2.1 (s, 3H), 2.95 (m, 1H), 5.05 (br.s, 1H), 7.4 (d, 2H), 7.6 (m, 3H), 8.05 (s, 1H), 8.3 (s, 1H), 10.4 (s, 1H), 11.9 (s, 1H) | 373 |
| 15 | 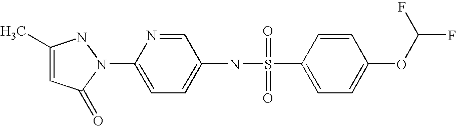 | benzenesulfonamide, 4- (difluoromethoxy)-N-[6- (2,5-dihydro-3-methyl-5- oxo-1H-pyrazol-1-yl)-3- pyridinyl]- | 1HNMR (DMSO, ppm): δ 2.1 (s, 3H), 5.1 (br.s, 1H), 7.3 (m, 3H), 7.6 (m, 1H), 7.8 (d, 2H), 8.05 (s, 2H), 10.5 (br.s, 1H), 11.9 (br.s, 1H) | 397 |
| 16 | 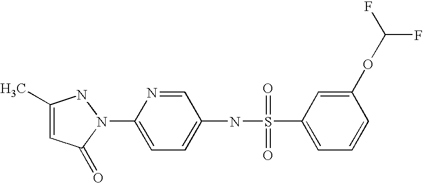 | benzenesulfonamide, 3- (difluoromethoxy)-N-[6- (2,5-dihydro-3-methyl-5- oxo-1H-pyrazol-1-yl)-3- pyridinyl]- | 1HNMR (DMSO, ppm): δ 2.1 (s, 3H), 5.05 (s, 1H), 7.3 (s, 1H), 7.6 (m, 5H), 8.05 (s, 1H), 8.3 (d, 1H), 10.45 (s, 1H), 11.9 (s, 1H) | 397 |
| 17 | 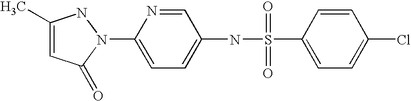 | benzenesulfonamide, 4- chloro-N-[6-(2,5-dihydro-3- methyl-5-oxo-1H-pyrazol-1- yl)-3-pyridinyl]- | 1HNMR (DMSO, ppm): δ 2.1 (s, 3H), 5.05 (s, 1H), 7.7 (m, 5H), 8.05 (s, 1H), 8.3 (d, 1H), 10.45 (br.s, 1H), 11.9 (s, 1H) | 365 |
| 18 | 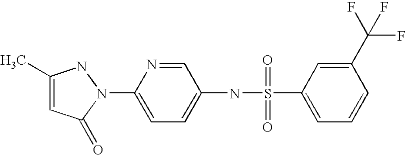 | benzenesulfonamide, N-[6- (2,5-dihydro-3-methyl-5- oxo-1H-pyrazol-1-yl)-3- pyridinyl]-3- (trifluoromethyl)- | 1HNMR (DMSO, ppm): δ 2.1 (s, 3H), 5.05 (s, 1H), 7.6 (m, 1H), 7.8 (m, 1H), 8.0 (m, 4H), 8.35 (d, 1H), 10.5 (s, 1H), 11.95 (s, 1H) | 399 |
| 19 | 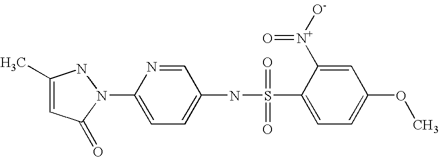 | benzenesulfonamide, N-[6- (2,5-dihydro-3-methyl-5- oxo-1H-pyrazol-1-yl)-3- pyridinyl]-4-methoxy-2- ntiro- | 1HNMR (DMSO, ppm): δ 2.15 (s, 3H), 4.05 (s, 3H,); 5.05 (s, 1H,); 7.5-7.7 (m, 1H,); 7.8-8.1 (m, 3H); 8.15 (s, 1H); 8.3 (d, 1H); 10.5 (s, 1H). 11.95 (s, 1H) | 406.1 |
| 20 | 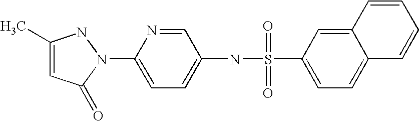 | 2-naphthalensulfonamide, N-[6- (2,5-dihydro-3- methyl-5-oxo-1H-pyrazol-1- yl)-3-pyridinyl]- | 1HNMR (DMSO, ppm): δ 2.15 (s, 3H); 5.05 (s, 1H,); 7.5-7.8 (m, 4H,); 7.95-8.2 (m, 4H); 8.2-8.3 (d, 1H); 8.35-8.5 (s, 1H); 10.5 (s, 1H). 11.95 (s, 1H) | 381.1 |
| 21 | 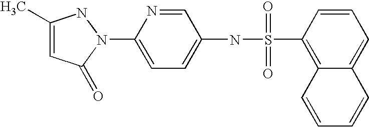 | 1-naphthalensulfonamide, N-[6- (2,5-dihydro-3- methyl-5-oxo-1H-pyrazol-1- yl)-3-pyridinyl]- | 1HNMR (DMSO, ppm): δ 2.15 (s, 3H); 5.05 (s, 1H,); 7.4-7.8 (m, 4H,); 7.9-8.05 (m, 1H); 8.1-8.3 (m, 4H); 8.6-8.75 (d, 1H); 10.8 (s, 1H). 11.85 (s, 1H) | 381.1 |
| 22 | 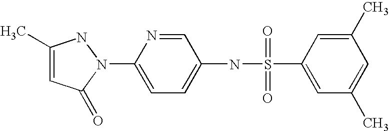 | benzenesulfonamide, N-[6- (2,5-dihydro-3-methyl-5- oxo-1H-pyrazol-1-yl)-3- pyridinyl]-3,5-dimethyl- | 1HNMR (DMSO, ppm): δ 2.1 (s, 3H), 2.3 (s, 6H), 5.05 (s, 1H), 7.25 (s, 1H), 7.35 (s, 2H), 7.6 (m, 1H), 8.05 (s, 1H), 8.25 (d, 1H), 10.3 (s, 1H), 11.9 (s, 1H) | 359 |
| 23 | 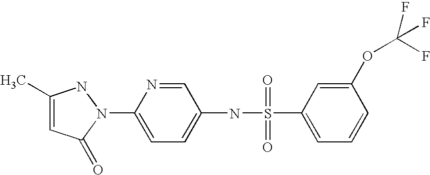 | benzenesulfonamide, N-[6- (2,5-dihydro-3-methyl-5- oxo-1H-pyrazol-1-yl)-3- pyridinyl]-3- (trifluoromethoxy)- | 1HNMR (DMSO, ppm): δ 2.1 (s, 3H), 5.05 (br.s, 1H), 7.7 (m, 5H), 8.05 (s, 1H), 8.3 (br.s, 1H), 10.5 (br.s, 1H), 11.9 (br.s, 1H) | 415 |
| 24 | 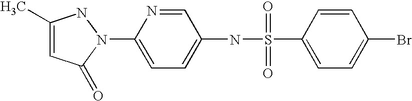 | benzenesulfonamide, 4- bromo-N-[6-(2,5-dihydro-3- methyl-5-oxo-1H-pyrazol-1- yl)-3-pyridinyl]- | 1HNMR (DMSO, ppm): δ 2.1 (s, 3H), 5.05 (s, 1H), 7.6 (m, 3H), 7.8 (s, 2H), 8.05 (s, 1H), 8.3 (d, 1H), 10.5 (br.s, 1H), 11.9 (br.s, 1H) | 411 |
| 25 | 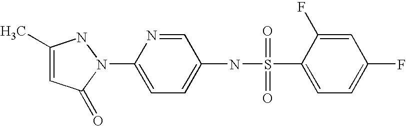 | benzenesulfonamide, N-[6- (2,5-dihydro-3-methyl-5- oxo-1H-pyrazol-1-yl)-3- pyridinyl]-2,4-difluoro- | 1HNMR (DMSO, ppm): δ 2.1 (s, 3H), 5.05 (s, 1H), 7.25 (t, 1H), 7.6 (m, 2H), 7.85 (m, 1H), 8.1 (s, 1H), 8.3 (d, 1H), 10.75 (br.s, 1H), 11.9 (s, 1H) | 367 |
| 26 | 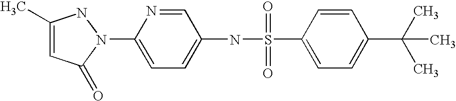 | benzenesulfonamide, N-[6- (2,5-dihydro-3-methyl-5- oxo-1H-pyrazol-1-yl)-3- pyridinyl]-4-(1,1- dimethylethyl- | 1HNMR (DMSO, ppm): δ 1.25 (s, 9H); 2.15 (s, 3H,); 5.05 (s, 1H,); 7.5-7.75 (m, 5H,); 8.15 (s, 1H); 8.3 (s, 1H); 10.45 (s, 1H). 11.95 (s, 1H) | 387.2 |
| 27 | 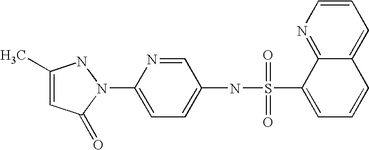 | 8-quinolinesulfonamide, N- [6-(2,5-dihydro-3-methyl-5- oxo-1H-pyrazol-1-yl)-3- pyridinyl]- | 1HNMR (DMSO, ppm): δ 2.15 (s, 3H); 5.05 (s, 1H,); 7.45-7.6 (d, 1H,); 7.65-7.8 (m, 2H); 7.9-8.1 (s, 2H); 8.25-8.4 (m, 2H); 8.5-8.6 (d, 1H); 9.15 (s, 1H) 10.35 (s, 1H); 11.75 (s, 1H) | 382.1 |
| 28 | 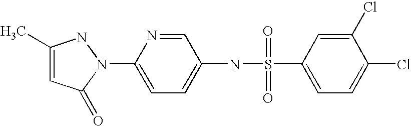 | benzenesulfonamide, 3,4- dichloro-N-[6-(2,5-dihydro- 3-methyl-5-oxo-1H-pyrazol- 1-yl)-3-pyridinyl]- | 1HNMR (DMSO, ppm): δ 2.1 (s, 3H), 5.05 (s, 1H), 7.6 (d, 2H), 7.85 (d, 1H), 7.95 (s, 1H), 8.1 (s, 1H), 8.3 (br.s, 1H), 10.55 (br.s, 1H), 11.9 (s, 1H) | 399 |
| 29 | 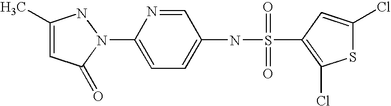 | 3-thiophenesulfonamide, 2,5-dichloro-N-[6-(2,5- dihydro-3-methyl-5-oxo- 1H-pyrazol-1-yl)-3- pyridinyl]- | 1HNMR (DMSO, ppm): δ 2.1 (s, 3H), 5.05 (s, 1H), 7.3 (s, 1H), 7.6 (s, 1H), 8.1 (s, 1H), 8.35 (d, 1H), 10.8 (br.s, 1H), 12 (s, 1H) | 405 |
| 30 | 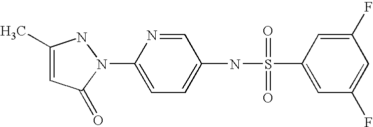 | benzenesulfonamide, N-[6- (2,5-dihydro-3-methyl-5- oxo-1H-pyrazol-1-yl)-3- pyridinyl]-3,5-difluoro- | 1HNMR (DMSO, ppm): δ 2.15 (s, 3H); 5.1 (s, 1H,); 7.45 (s, 2H,); 7.5-7.75 (m, 2H); 8.15 (s, 1H); 8.3-8.4 (d, 1H); 10.6 (s, 1H). 11.95 (s, 1H) | 367.1 |
| 31 | 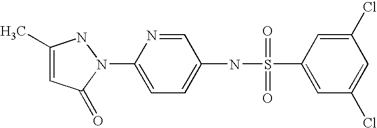 | benzenesulfonamide, 3,5- dichloro-N-[6-(2,5-dihydro- 3-methyl-5-oxo-1H-pyrazol- 1-yl)-3-pyridinyl]- | 1HNMR (DMSO, ppm): δ 2.15 (s, 3H); 5.1 (s, 1H,); 7.45-7.8 (m, 3H,); 7.9-8.15 (d, 2H); 8.3-8.45 (d, 1H); 10.6 (s, 1H). 11.95 (s, 1H) | 401 |
| 32 | 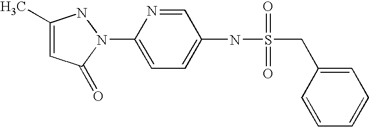 | benzenemethanesulfonamide, N-[6- (2,5-dihydro-3- methyl-5-oxo-1H-pyrazol-1- yl)-3-pyridinyl]- | 1HNMR (DMSO, ppm): δ 2.15 (s, 3H); 4.55 (s, 2H,); 5.05 (s, 1H,); 7.2-7.45 (m, 5H,); 7.55-7.8 (m, 1H); 8.1-8.2 (d, 1H); 8.3-8.45 (d, 1H); 9.95 (s, 1H). 11.95 (s, 1H) | 345.1 |
| 33 | 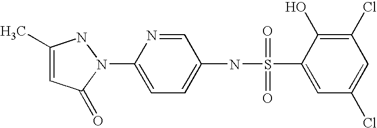 | benzenesulfonamide, 3,5- dichloro-N-[6-(2,5-dihydro- 3-methyl-5-oxo-1H-pyrazol- 1-yl)-3-pyridinyl]-2- hydroxy- | 1HNMR (DMSO, ppm): δ 2.1 (s, 3H), 5.1 (br.s, 1H), 7.6 (s, 2H), 7.85 (s, 1H), 8.1 (s, 2H), 10.5 (br.s, 1H), 11.2 (br.s, 1H), 11.9 (br.s, 1H) | 415 |
| 34 | 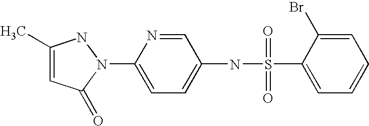 | benzenesulfonamide, 2- bromo-N-[6-(2,5-dihydro-3- methyl-5-oxo-1H-pyrazol-1- yl)-3-pyridinyl]- | 1HNMR (DMSO, ppm): δ 2.1 (s, 3H), 5.1 (br.s, 1H), 7.5 (s, 3H), 7.75 (d, 1H), 8.0 (s, 3H), 11.2 (br.s, 1H), 11.9 (br.s, 1H) | 411 |
| 35 | 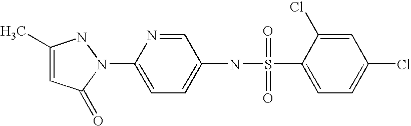 | benzenesulfonamide, 2,4- dichloro-N-[6-(2,5-dihydro- 3-methyl-5-oxo-1H-pyrazol- 1-yl)-3-pyridinyl]- | 1HNMR (DMSO, ppm): δ 2.1 (s, 3H), 5.1 (br.s, 1H), 7.6 (m, 2H), 7.85 (s, 1H), 8.0 (d, 1H), 8.3 (br.s, 1H), 10.85 (br.s, 1H), 11.9 (br.s, 1H) | 399 |
| 36 | 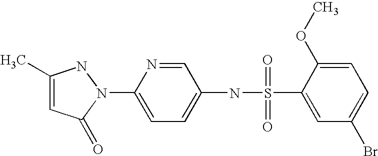 | benzenesulfonamide, 5- bromo-N-[6-(2,5-dihydro-3- methyl-5-oxo-1H-pyrazol-1- yl)-3-pyridinyl]-2-methoxy | 1HNMR (DMSO, ppm): δ 2.1 (s, 3H), 3.9 (s, 3H), 5.1 (s, 1H), 7.2 (d, 1H), 7.55 (d, 1H), 7.75 (d, 2H), 8.1 (s, 1H), 8.3 (d, 1H), 10.25 (s, 1H), 11.9 (s, 1H) | 441 |
| 37 | 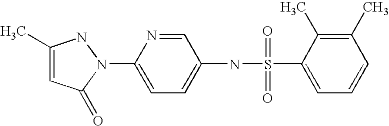 | benzenesulfonamide, N-[6- (2,5-dihydro-3-methyl-5- oxo-1H-pyrazol-1-yl)-3- pyridinyl]-3,4-dimethyl- | 1HNMR (DMSO, ppm): δ 2.15 (s, 3H); 2.25 (s, 6H); 5.05 (s, 1H,); 7.25-7.35 (d, 1H,); 7.4-7.5 (d, 1H); 7.5-7.7 (m, 2H); 8.15 (s, 1H); 8.25 (s, 1H): 10.3 (s, 1H). 11.95 (s, 1H) | 359 |
| 38 | 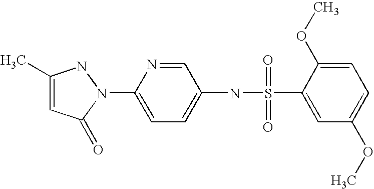 | benzenesulfonamide, N-[6- (2,5-dihydro-3-methyl-5- oxo-1H-pyrazol-1-yl)-3- pyridinyl]-2,5-dimethoxy- | 1HNMR (DMSO, ppm): δ 2.1 (s, 3H), 3.7 (s, 3H), 3.8 (s, 3H), 5.1 (s, 1H), 7.2 (m, 3H), 7.55 (m, 1H), 8.1 (s, 1H), 8.25 (d, 1H), 10.1 (s, 1H), 11.9 (s, 1H) | 391 |
| 39 | 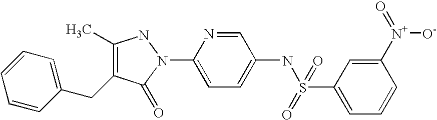 | N-[6-(4-benzyl-3-methyl-5- oxo-2,5-dihydro-1H- pyrazol-1-yl)pyridin-3-yl]- 3-nitrobenzenesulfonamide | 1HNMR (CDCl3, ppm): δ 2.07 (s, 3H, CH3); 3.85 (s, 2H, CH2); 6.90 (dd, 1H, J = 3.01, 8.69 Hz); 7.12-7.28 (m, 8H); 7.58-7.61 (m, 2H); 7.99 (d, 1H); 8.35 (d, 1H,); 8.42 (s, 1H,). | 466 |
| 40 | 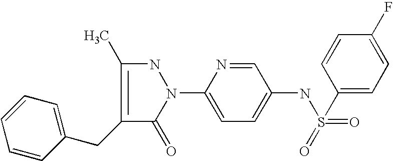 | N-[6-(4-benzyl-3-methyl-5- oxo-2,5-dihydro-1H- pyrazol-1-yl)pyridin-3-yl]- 4-fluorobenzenesulfonamide | 1HNMR (DMSO, ppm): δ 2.50 (s, 3H, —CH3); 3.50 (s, 2H, —CH2—), 7.31 (m, 5H, Aro.), 7.43 (t, 2H, Aro.), 7.58 (d, 1H, Aro.), 7.77 (t, 2H, Aro.), 8.03 (s, 1H, Aro.), 8.35 (t, 1H, Aro.), 10.37 (s,1H, Aro.), 11.60 (s, 1H, Aro.) | 439 |
| 41 | 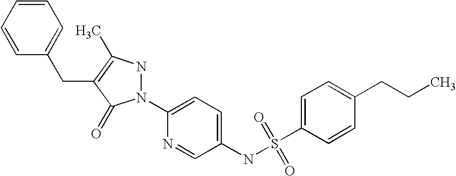 | benzenesulfonamide, N-[6- [2,5-dihydro-3-methyl-5- oxo-4-(phenylmethyl)-1H- pyrazol-1-yl]-3-pyridinyl]- 4-propyl- | 1HNMR (DMSO, ppm): δ 0.7-0.8 (t, 3H), 1.4-1.7 (m, 2H), 2.1 (s, 3H), 2.5-2.7 (t, 2H), 3.5 (s, 2H), 7.1.-7.4 (m, 7H), 7.5-7.7 (m, 3H), 8.1 (s, 1H), 8.2-8.4 (d, 1H), 10.2-10.4 (br s 1H), 11.6 (s, 1H) | 463 |
| 42 | 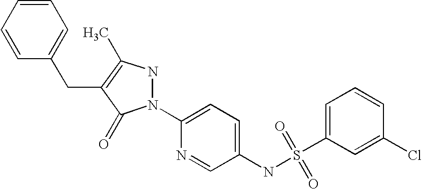 | benzenesulfonamide, 3- chloro-N-[6-[2,5-dihydro-3- methyl-5-oxo-4- (phenylmethyl)-1H-pyrazol- 1-yl]-3-pyridinyl]- | 1HNMR (CDCl3, ppm): δ 2.2 (s, 3H), 3.7 (s, 2H), 7.1-7.5 (m, 5H), 7.4-7.5 (m, 1H), 7.5-7.7 (m, 3H), 7.8 (s, 1H), 7.9-8 (d, 1H), 8.1 (s, 1H) | 455 |
| 43 | 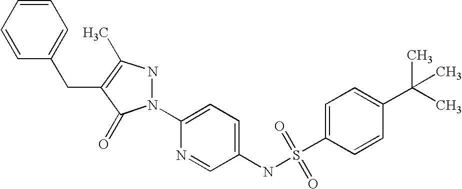 | benzenesulfonamide, N-[6- [2,5-dihydro-3-methyl-5- oxo-4-(phenylmethyl)-1H- pyrazol-1-yl]-3-pyridinyl]- 4-(1,1-dimethylethyl)- | 1HNMR (DMSO, ppm): δ 1.3 (s, 9H), 2.1 (s, 3H), 3.5 (s, 2H), 7.1-7.4 (m, 5H), 7.5-7.7 (m, 5H), 8.1 (s, 1H), 8.3-8.4 (d, 1H), 10.4 (s, 1H), 11.6 (s, 1H) | 477 |
| 44 | 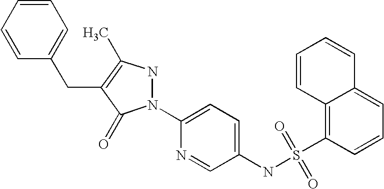 | 1-naphthalensulfonamide, N-[6- [2,5-dihydro-3- methyl-5-oxo-4- (phenylmethyl)-1H-pyrazol- 1-yl]-3-pyridinyl]- | 1HNMR (DMSO, ppm): δ 2.1 (s, 3H), 3.5 (s, 2H), 7.1-7.3 (m, 5H), 7.4-7.8 (m, 4H), 7.9-8.3 (m, 5H), 8.7-8.8 (d, 1H), 10.8 (s, 1H), 11.5 (s, 1H) | 471 |
| 45 | 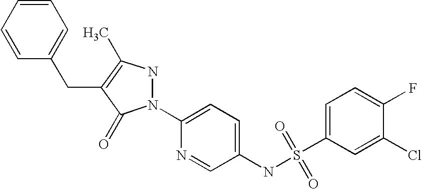 | benzenesulfonamide, 3- chloro-N-[6-[2,5-dihydro-3- methyl-5-oxo-4- (phenylmethyl)-1H-pyrazol- 1-yl]-3-pyridinyl]-4-fluoro- | 1HNMR (DMSO, ppm): δ 2.1 (s, 3H), 3.5 (s, 2H), 7.1-7.3 (m, 5H), 7.5-7.7 (m, 3H), 7.9-8.0 (m, 1H), 8.1 (s, 1H), 8.3-8.4 (d, 1H), 10.5 (s, 1H), 11.6 (s, 1H) | 473 |

### EXAMPLE 46

### 5 4-methyl-N-[6-(3-methyl-5-oxo-2,5-dihydro-1H-pyrazol-1-yl)pyridin-3-yl]benzamide

The intermediate from step C in Example 1 above was used here.

### Step D: N-[6-(2,5-dihydro-3-methyl-5-oxo-1H-pyrazol-1-yl-3-pyridinyl]-4-methyl benzamide

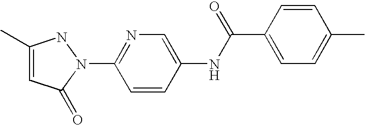

In a 10 mL thermal reactor tube, 4-methylbenzoic acid (0.13 g, 1.0 mmol), EDCI.HCl (0.23 g, 1.2 mmol), 4-dimethylaminopyridine (0.15 g, 1.2 mmol) were mixed together in dichloromethane (5 mL). The mixture was stirred for 30 minutes to afford a clear solution. To the stirred solution, intermediate from step C (0.19 g, 1 mmol) was added and the reaction mixture was stirred for 15 h. Precipitated solid was filtered in vacuo and washed with cold dichloromethane. The crude solid was suspended in ethyl acetate (10 mL) and sonicated for few minutes. Filtered, washed with ethyl acetate and dried in vacuo to afford the title compound as off white solid (0.11 g, 36%).

¹H NMR: (DMSOD6, δ ppm): 2.15 (s, 3H), 2.40 (s, 3H), 5.09*, 5.55* (br s, 1H), 7.35 (d, 2H), 7.70-7.88*, 8.15-8.50* (m, 2H), 7.91 (d, 2H), 8.85 (s, 1H), 10.40 (br s, 1H), 12.00 (br s, 1H). (* rotamers)

MS (ES+) 308.1

The compounds set out below were prepared in the same way as in Example 46, using the appropriate starting materials.

| Ex- | | | | |
|---|---|---|---|---|
| am- | | | | MASS |
| ple | Structure | Name | NMR | (ES+) |
| 47 | 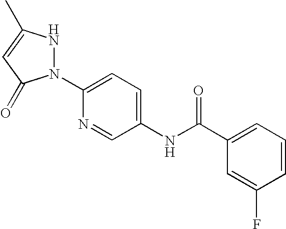 | 3-fluoro-N-[6-(3-methyl-5- oxo-2,5-dihydro-1H- pyrazol-1-yl)pyridin-3- yl]benzamide | (DMSOD6, ppm): δ 2.15 (s, 3H), 5.10*, 5.50* (br s, 1H), 7.40-7.55 (m, 1H), 7.55-7.69 (m, 1H), 7.70-7.95 (m, 2H), 8.22 (dd, 1H), 8.35*, 8.45* (d, 1H), 8.80*, 8.89* (s, 1H), 5.55*, 5.65* (br s, 1H), 12.00 (br s, 1H) | 312.1 |
| 48 | 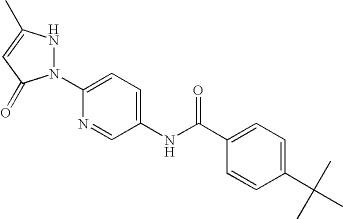 | 4-tert-butyl-N-[6-(3-methyl- 5-oxo-2,5-dihydro-1H- pyrazol-1-yl)pyridin-3- yl]benzamide | (DMSOD6, ppm): δ 1.31 (s, 9H), 2.19 (s, 3H), 5.10*, 5.50* (br s, 1H), 7.58 (d, 2H), 7.95 (d, 2H), 8.15-8.55 (br m, 2H), 8.88 (s, 1H), 10.50 (br, s, 1H), 12.00 (br s, 1H) | 350.1 |
| 49 | 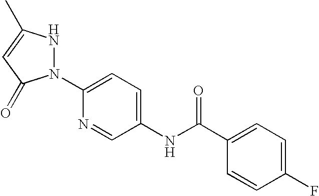 | 4-fluoro-N-[6-(3-methyl-5- oxo-2,5-dihydro-1H- pyrazol-1-yl)pyridin-3- yl]benzamide | (DMSOD6, ppm): δ 2.15 (s, 3H), 5.10*, 5.50* (br s, 1H), 7.40 (t, 2H), 7.75*, 8.21* (br s, 1H), 8.00-8.15 (m, 2H), 8.30-8.50 (m, 1H), 8.89 (s, 1H), 10.55 (br s, 1H), 12.05 (br s, 1H) | 312.1 |
| 50 | 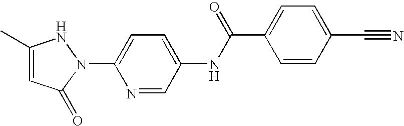 | 4-cyano-N-[6-(3-methyl-5- oxo-2,5-dihydro-1H- pyrazol-1-yl)pyridin-3- yl]benzamide | (DMSOD6, ppm): δ 2.18 (s, 3H), 5.10*, 5.45* (br s, 1H), 7.85*, 8.25* (br s, 1H), 8.05 (d, 2H), 8.15 (d, 2H), 8.30-8.50 (br m, 1H), 8.95 (s, 1H), 10.75*, 10.85* (br s, 1H), 12.05 (br s, 1H) | 319.1 |
| 51 | 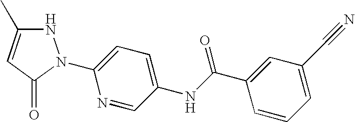 | 3-cyano-N-[6-(3-methyl-5- oxo-2,5-dihydro-1H- pyrazol-1-yl)pyridin-3- yl]benzamide | (DMSOD6, ppm): δ 2.10 (s, 3H), 5.05*, 5.40* (br s, 1H), 7.75 (t,, 1H), 8.05 (d, 1H), 8.15*, 8.25* (d, 2H), 8.30*, 8.40 (s, 2H), 8.75*, 8.85* (s, 1H), 10.60*, 10.75* (br s, 1H), 12.00 (br s, 1H) | 319.1 |
| 52 | 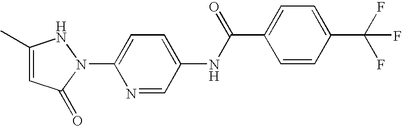 | N-[6-(3-methyl-5-oxo-2,5- dihydro-1H-pyrazol-1- yl)pyridin-3-yl]-4- (trifluoromethyl)benzamide | (DMSOD6, ppm): δ 2.10 (s, 3H), 5.00*, 5.40* (br s, 1H), 7.70*, 8.18* (d, 1H), 7.90 (d, 2H), 8.15 (d, 2H), 8.25-8.45 (m, 1H), 8.75*, 8.85* (s, 1H), 10.65*, 10.75* (br s, 1H), 12.00 (br s, 1H) | 362.1 |
| 53 | 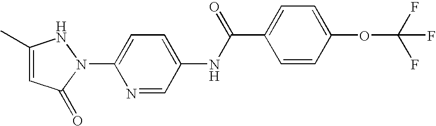 | N-[6-(3-methyl-5-oxo-2,5- dihydro-1H-pyrazol-1- yl)pyridin-3-yl]-4- (trifluoromethoxy)benzamide | (DMSOD6, ppm): δ 2.15 (s, 3H), 5.10*, 5.45* (br s, 1H), 7.58*, 7.75* (d, 1H), 8.10* 8.20* (d, 3H), 8.35*, 8.45* (d, 1H), 8.75*, 8.85* (s, 1H), 10.55*, 10.70* (br s, 1H), 12.00 (br s, 1H) | 378.1 |
| 54 | 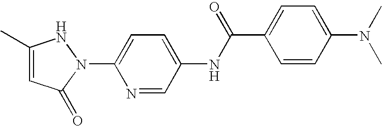 | 4-(dimethylamino)-N-[6-(3- methyl-5-oxo-2,5-dihydro- 1H-pyrazol-1-yl)pyridin-3- yl]benzamide | (DMSOD6, ppm): δ 2.05*, 2.15* (s, 3H), 2.95 (s, 6H), 5.00*, 5.40* (br s, 1H), 6.72 (d, 2H), 7.65*, 8.15* (d, 1H), 7.85 (d, 2H), 8.20-8.35 (m, 1H), 8.75*, 8.80* (s, 1H), 10.00*, 10.18* (br s, 1H), 11.90*, 12.00* (br s, 1H) | 337.2 |
| 55 | 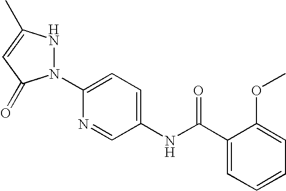 | 2-methoxy-N-[6-(3- methyl-5-oxo-2,5-dihydro- 1H-pyrazol-1-yl)pyridin-3- yl]benzamide | (DMSOD6, ppm): δ 2.11*, 2.19* (s, 3H), 3.90 (s, 3H), 5.09*, 5.45* (br s, 1H), 7.05 (t, 1H), 7.20 (d, 1H), 7.50 (t, 1H), 7.65 (d, 1H), 7.75*, 8.15 (d, 1H), 8.30*, 8.40* (d, 1H), 8.75*, 8.85* (s, 1H), 10.30*, 10.45* (br s, 1H), 12.00 (br s, 1H) | 324.1 |
| 56 | 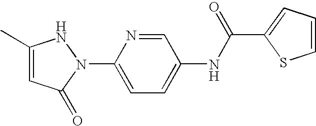 | N-[6-(3-methyl-5-oxo-2,5- dihydro-1H-pyrazol-1- yl)pyridin-3-yl]-thiophene-2- carboxamide | (DMSOD6, ppm): δ 2.10*, 2.19* (s, 3H), 5.09*, 5.48* (br s, 1H), 7.25 (t, 1H), 7.75*, 8.15* (d, 1H), 7.90 (d, 1H), 8.05 (d, 1H), 8.30*, 8.45* (d, 1H), 8.78*, 8.85* (s, 1H), 10.48*, 10.58* (br s, 1H), 12.00 (br s, 1H) | 300.1 |
| 57 | 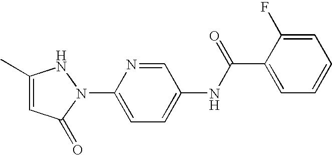 | 2-fluoro-N-[6-(3-methyl-5- oxo-2,5-dihydro-1H- pyrazol-1-yl)pyridin-3- yl]benzamide | (DMSOD6, ppm): δ 2.15 (s, 3H), 5.09*, 5.48* (br s, 1H), 7.38 (q, 2H), 7.60 (q, 1H), 7.75 (t, 1H), 8.10-8.50 (m, 2H), 8.85 (s, 1H), 10.65*, 10.75* (br s, 1H), 12.00 (br s, 1H) | 337.2 |
| 58 | 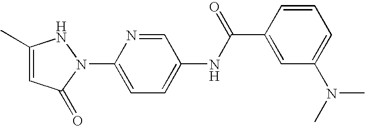 | 3-(dimethylamino)-N-[6-(3- methyl-5-oxo-2,5-dihydro- 1H-pyrazol-1-yl)pyridin-3- yl]benzamide | (DMSOD6, ppm): δ 2.11*, 2.18* (s, 3H), 3.00 (s, 6H), 5.10*, 5.48* (br s, 1H), 6.95 (d, 1H), 7.25 (d, 2H), 7.35 (t, 1H), 7.75*, 8.20* (d, 1H), 8.35*, 8.45* (d, 1H), 8.85*, 8.89* (s, 1H), 10.35*, 10.40* (br s, 1H), 12.00* (br s, 1H) | 312.1 |
| 59 | 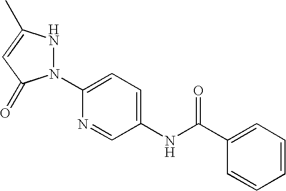 | N-[6-(3-methyl-5-oxo-2,5- dihydro-1H-pyrazol-1- yl)pyridin-3-yl]-benzamide | (DMSOD6, ppm): δ 2.15*, 2.20* (s, 3H), 5.10*, 5.48* (br s, 1H), 7.50-7.70 (m, 3H), 7.75*, 8.22* (d, 1H), 8.00 (d, 2H), 8.40*, 8.45* (d, 1H), 8.81*, 8.89* (s, 1H), 10.50*, 10.62* (br s, 1H), 12.00* (br s, 1H) | 294.1 |
| 60 | 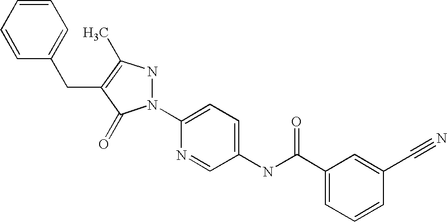 | benzamide, 3-cyano-N-[6- [2,5-dihydro-3-methyl-5- oxo-4-(phenylmethyl)-1H- pyrazol-1-yl]-3-pyridinyl]- | 1HNMR (DMSO, ppm): δ 2.2 (s, 3H), 3.6 (s, 2H), 7.1-7.4 (m, 5H), 7.7-7.9 (t, 1H), 8.1-8.2 (d, 2H), 8.2-8.3 (m, 2H), 8.4-8.6 (m, 2H), 10.7 (s, 1H), 11.7 (s, 1H) | |
| 61 | 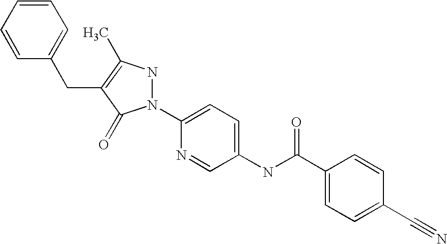 | benzamide, 4-cyano-N-[6- [2,5-dihydro-3-methyl-5- oxo-4-(phenylmethyl)-1H- pyrazol-1-yl]-3-pyridinyl]- | 1HNMR (DMSO, ppm): δ 2.2 (s, 3H), 3.6 (s, 2H), 7.1-7.3 (m, 5H), 8.0-8.2 (m, 4H), 8.2-8.3 (d, 1H), 8.4-8.6 (d, 1H), 8.8 (s, 1H), 10.7 (s, 1H), 11.7 (s, 1H) | |

### EXAMPLE 62

### 1,2-dihydro-5-methyl-2-{6-[[4-(trifluoromethoxy)phenyl]methoxy]-3-pyridazinyl}-3H-pyrazol-3-one

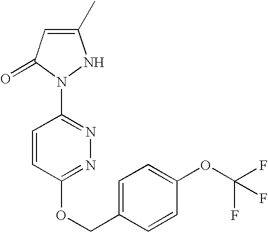

### Step A: (3Z)-3-[(6-chloro-3-pyridazinyl)hydrazono]-butanoic acid, ethyl ester

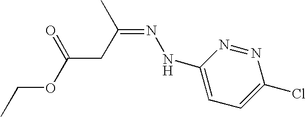

In a 100 mL round bottom flask, ethyl acetoacetate (3.24 g, 3.15 mL, 24.90 mmol) was added to the stirred suspension of 3-chloro-6-hydrazinopyridazine (3 g, 20.75 mmol) in ethanol (25 mL). The mixture became very thick and difficult to stir after few minutes. It was kept at RT for 1 h. The thick suspension was diluted with chilled ethanol (20 mL) and filtered at pump. The solid was washed with chilled ethanol (20 mL) and dried in vacuo to afford the title compound as a yellowish brown crystalline solid (3.00 g, 56%). Additional crop (0.5 g) could be recovered from the filtrate after concentration to small volume.

1H NMR (CDCl₃): δ 1.30 (t, 3H), 2.15 (s, 3H), 3.38 (s, 2H), 4.22 (q, 2H), 7.28 (s, 1H), 7.40 (d, 1H), 7.62 (d, 1H), 8.80 (br s, 1H).

### Step B: 2-(6-chloro-3-pyridazinyl)-1,2-dihydro-5-methyl-3H-pyrazol-3-one

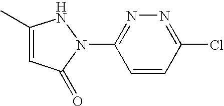

In a 100 mL round bottom flask, KOtBu (1.13 g, 10.11 mmol) was added in a single lot to the solution of the intermediate from Step A (2.18 g, 8.43 mmol) in ethanol (25 mL). The yellow coloured solution immediately turned dark green and a dark green coloured precipitate started appearing. The reaction mixture was stirred RT for 3 h. The solvent was removed in vacuo. The residue was taken up in water (25 mL) and extracted with ether (30 mL). The aqueous layer was cooled and acidified with glacial acetic acid. A buff coloured precipitate was observed. It was filtered at pump and washed with cold water. Dried in vacuo to afford the title compound as buff colored solid (1.78 g, 93%).

1H NMR (DMSOD₆, ppm): δ 2.20 (s, 3H), 5.18 (s, 1H), 7.98 (d, 1H), 8.78 (d, 1H), 12.55 (br s, 1H).

### Step C: 1,2-dihydro-5-methyl-2-{6-[[4-(trifluoromethoxy)phenyl]methoxy]-3-pyridazinyl}-3H-pyrazol-3-one

In a 20 mL thermal reactor tube, intermediate from step B (0.15 g, 0.71 mmol), 4-(trifluoromethoxy)benzenemethanol (0.55 g, 0.29 mmol), KOtBu (0.32 g, 0.29 mmol) were mixed in dry THF (10 mL) and the mixture was refluxed for 15 h. The reaction mixture was then diluted with water (20 mL) and extracted with ether (3×20 mL). The aqueous layer was then acidified with glacial acetic acid. The precipitated solid was filtered in vacuo, washed with water and dried. The crude solid (˜0.25 g) was purified by chromatography on silica gel column using 3% methanol in dichloromethane as eluent followed by recrystallization from methanol to afford the title compound as a white crystalline solid (0.09 g, 35%).

¹H NMR (DMSOD6, δ ppm): 2.19 (s, 3H), 5.15 (s, 1H), 5.65 (s, 2H), 7.50 (d, 2H), 7.53 (d, 1H), 7.62 (d, 2H), 8.64 (d, 1H), 12.38 (br s, 1H)

The compounds set out below were prepared in the same way as in Example 62, using the appropriate starting materials.

| | | | | MASS |
|---|---|---|---|---|
| Example | Structure | Name | NMR | (ES+) |
| 63 | 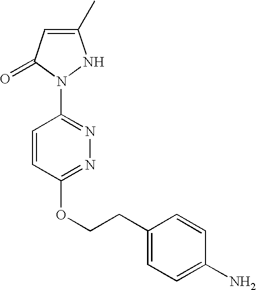 | 2-{6-[2-(4- aminophenyl)ethoxy] pyridazin-3-yl}-5-methyl- 1,2-dihydro-3H- pyrazol-3-one | (DMSOD6, ppm): δ 2.19 (s, 3H), 3.15 (t, 2H), 4.50 (t, 2H), 5.10 (s, 1H), 6.50 (d, 2H), 6,98 (d, 2H), 7.35 (d, 1H), 8.64 (d, 1H), 12.38 (br s, 1H) | 311.3 |
| 64 | 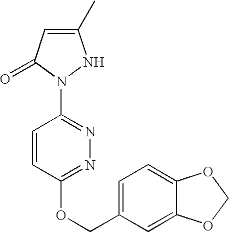 | 2-[6-(1,3-benzodioxol- 5-ylmethoxy)pyridazin- 3-yl]-5-methyl-1,2- dihydro-3H-pyrazol-3- one | (DMSOD6, ppm): δ 2.19 (s, 3H), 5.10 (s, 1H), 5.40 (s, 2H), 6.05 (s, 2H), 6.91 (d, 1H), 7.02 (d, 1H), 7.10 (s, 1H), 7.50 (d, 1H), 8.64 (d, 1H), 12.38 (br s, 1H) | 326.3 |
| 65 | 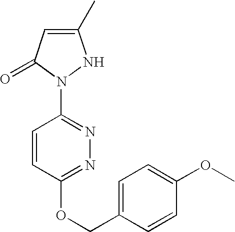 | 2-{6-[(4- methoxybenzyl)oxy] pyridazin-3-yl}-5-methyl- 1,2-dihydro-3H- pyrazol-3-one | (DMSOD6, ppm): δ 2.19 (s, 3H), 3.78 (s, 3H), 5.10 (s, 1H), 5.40 (s, 2H), 6.95 (d, 2H), 7.35 (d, 1H), 7.45 (d, 2H), 8.64 (d, 1H), 12.38 (br s, 1H) | 312.3 |
| 66 | 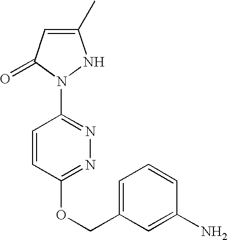 | 2-{6-[(3- aminobenzyl)oxy] pyridazin-3-yl}-5-methyl- 1,2-dihydro-3H- pyrazol-3-one | (DMSOD6, ppm):δ 2.15 (s, 3H), 5.10 (s, 1H), 5.20 (br s, 2H), 5.35 (s, 2H), 6.50 (d, 1H), 6.60 (d, 1H), 6.65 (s, 1H), 7.00 (t, 1H), 7.40 (d, 1H), 8.65 (d, 1H), 12.38 (br s, 1H) | 297.3 |
| 67 | 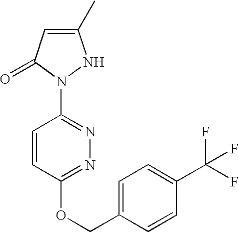 | 5-methyl-2-(6-{[4- (trifluoromethyl)benzyl] oxy}pyridazin-3-yl)- 1,2-dihydro-3H- pyrazol-3-one | (DMSOD6, ppm): δ 2.15 (s, 3H), 5.10 (s, 1H), 5.60 (s, 2H), 7.48 (d, 1H), 7.65-7.82 (m, 4H), 8.65 (d, 1H), 12.38 (br s, 1H) | 350.3 |
| 68 | 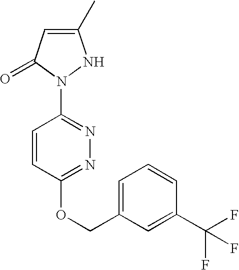 | 5-methyl-2-(6-{[3- (trifluoromethyl)benzyl] oxy}pyridazin-3-yl)- 1,2-dihydro-3H- pyrazol-3-one | (DMSOD6, ppm): δ 2.19 (s, 3H), 5.11 (s, 1H), 5.60 (s, 2H), 7.45 (d, 1H), 7.65 (t, 1H), 7.70 (d, 1H), 7.80 (d, 1H), 7.89 (s, 1H), 8.68 (d, 1H), 12.38 (br s, 1H) | 350.3 |
| 69 | 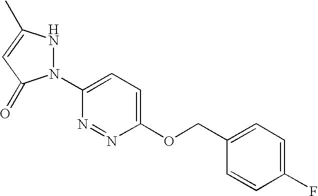 | 2-{6-[(4- fluorobenzyl)oxy] pyridazin-3-yl}-5-methyl- 1,2-dihydro-3H- pyrazol-3-one | (DMSOD6, ppm): δ 2.19 (s, 3H), 5.12 (s, 1H), 5.49 (s, 2H), 7.21 (t, 2H), 7.41 (d, 1H, 7.59 (dd, 2H), 8.68 (d, 1H), 12.38 (br s, 1H) | 300.1 |
| 70 | 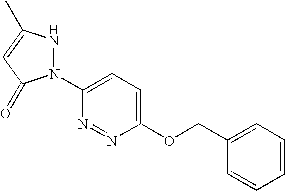 | 2-[6- (benzyloxy)pyridazin- 3-yl]-5-methyl-1,2- dihydro-3H-pyrazol-3- one | (DMSOD6, ppm): δ 2.15 (s, 3H), 5.11 (br s, 1H), 5.51 (s, 2H), 7.30-7.48 (m, 4H), 7.51 (d, 2H), 8.68 (d, 1H), 12.38 (br s, 1H) | 282.1 |
| 71 | 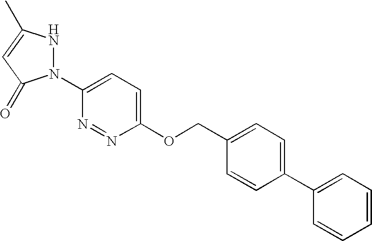 | 2-[6-(1,1′-biphenyl-4- ylmethoxy)pyridazin-3- yl]-5-methyl-1,2- dihydro-3H-pyrazol-3- one | (DMSOD6, ppm): δ 2.19 (s, 3H), 5.11 (s, 1H), 5.55 (s, 2H), 7.30-7.45 (m, 4H), 7.60 (d, 2H), 7.65-7.80 (m, 4H), 8.68 (d, 1H), 12.38 (br s, 1H) | 358.1 |
| 72 | 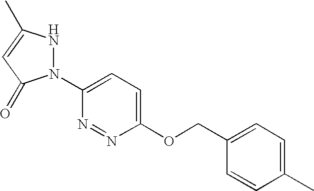 | 5-methyl-2-{6-[(4- methylbenzyl)oxy] pyridazin-3-yl}- 1,2-dihydro-3H- pyrazol-3-one | (DMSOD6, ppm): δ 2.19 (s, 3H), 2.30 (s. 3H), 5.12 (s, 1H), 5.48 (s, 2H), 7.20 (d, 2H), 7.40 (d, 3H), 8.68 (d, 1H), 12.38 (br s, 1H) | 296.1 |
| 73 | 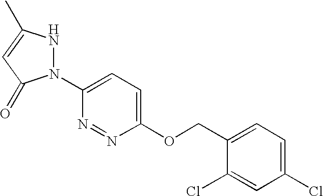 | 2-{6-[(2,4- dichlorobenzyl)oxy] pyridazin-3-yl}-5-methyl- 1,2-dihydro-3H- pyrazol-3-one | (DMSOD6, ppm): δ 2.19 (s, 3H), 5.12 (br s, 1H), 5.48 (s, 2H), 7.40-7.52 (m, 2H), 7.65 (d, 1H), 7.71 (d, 1H), 8.69 (d, 1H), 12.38 (b, 1H) | 351.2 |
| 74 | 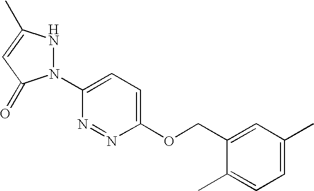 | 2-{6-[(2,5- dimethylbenzyl)oxy] pyridazin-3-yl}-5-methyl- 1,2-dihydro-3H- pyrazol-3-one | (DMSOD6, ppm): δ 2.19 (s, 3H), 2.29 (s, 3H), 2.31 (s, 3H), 5.11 (s, 1H), 5.48 (s, 2H), 7.05 (d, 1H), 7.11 (d, 1H), 7.29 (s, 1H), 7.45 (d, 1H), 8.68 (d, 1H), 12.38 (br s, 1H) | 310.1 |
| 75 | 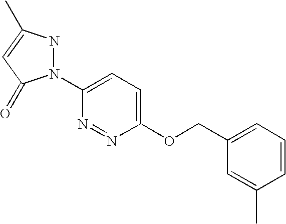 | 5-methyl-2-{6-[(3- methylbenzyl) oxy]pyridazin-3-yl}- 1,2-dihydro-3H- pyrazol-3-one | (DMSOD6, ppm): δ 2.19 (s, 3H), 2.31 (s, 3H), 5.11 (s, 1H), 5.48 (s, 2H), 7.10-7.21 (m, 1H), 7.25-7.37 (m, 3H), 7.41 (d, 1H), 8.68 (d, 1H), 12.38 (br s, 1H) | 296.1 |
| 76 | 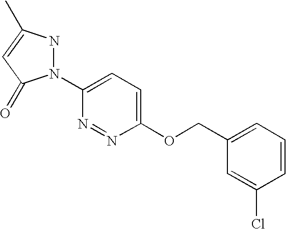 | 2-{6-[(3- chlorobenzyl)oxy] pyridazin-3-yl}-5-methyl- 1,2-dihydro-3H- pyrazol-3-one | (DMSOD6, ppm): δ 2.19 (s, 3H), 5.11 (s, 1H), 5.50 (s, 2H), 7.35-7.52 (m, 4H), 7.60 (s, 1H), 8.68 (d, 1H), 12.38 (br s, 1H) | 316.7 |
| 77 | 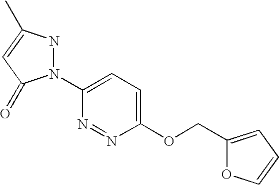 | 2-[6-(2- furylmethoxy)pyridazin- 3-yl]-5-methyl-1,2- dihydro-3H-pyrazol-3- one | (DMSOD6, ppm): δ 2.19 (s, 3H), 5.11 (br s, 1H), 5.48 (s, 2H), 6.58 (s, 1H), 6.65 (s, 1H), 7.30-7.50 (m, 1H), 7.82 (s, 1H), 8.68 (d, 1H), 12.38 (b, 1H) | 272.1 |

1. A compound of the formula (I) 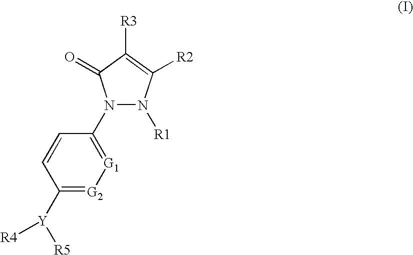
wherein G₁ and G₂ are independently selected from C or N and the aromatic ring comprising them is further optionally substituted by one or two C₁₋₆ alkyl groups,
Y is O, N or C═O,
R1 is H or C₁₋₆ alkyl,
R2 is H or C₁₋₆ alkyl; C₆₋₁₀ aryl-C₁₋₆ alkyl-, C₆₋₁₀ heteroaryl-C₁₋₆ alkyl-, C₁₋₆ alkoxy, C₆₋₁₀ aryl-C₁₋₆ alkoxy-, C₆₋₁₀ heteroaryl-C₁₋₆ alkoxy-, or —N substituted by one or two C₁₋₄ alkyl groups;
R3 is H, C₁₋₆ alkyl, C₆₋₁₀ aryl-C₁₋₆ alkyl-, or C₆₋₁₀ heteroaryl-C₁₋₆ alkyl-, C₁₋₆ alkoxy, C₆₋₁₀ aryl-C₁₋₆ alkoxy-, C₆₋₁₀ heteroaryl-C₁₋₆ alkoxy-, or —N substituted by one or two C₁₋₄ alkyl groups;
R4 is H or C₁₋₆ alkyl, except where Y is O or C═O then R4 is absent,
R5 is C₁₋₆ alkyl, C₅₋₁₀ aryl, C₅₋₁₀ heteroaryl, C₅₋₁₀ aryl-C₁₋₆ alkyl-, C₅₋₁₀ heteroaryl-C₁₋₆ alkyl, SO₂—C₅₋₁₀ aryl or SO₂—C₅₋₁₀ heteroaryl, C═O—C₅₋₁₀ aryl or C═O—C₅₋₁₀ heteroaryl, and when Y is C═O then additionally —NH—C₅₋₁₀ aryl or —NH—C₅₋₁₀ heteroaryl,
wherein heteroaryl comprises 1-3 heteroatoms independently selected from N,O, or S and wherein each aryl or heteroaryl group is optionally substituted by 1-3 groups independently selected from C₁₋₆ alkyl, C₁₋₆ alkoxy, difluoromethyl, trifluoromethyl, difluoromethoxy, trifluoromethoxy, halogen, hydroxy, NO₂, amino, di-C₁₋₆ alkylamino, phenyl or CN,
or a pharmaceutically acceptable salt or an in vivo hydrolysable ester thereof.

2. A compound as claimed in claim 1 or a pharmaceutically acceptable salt or an in vivo hydrolysable ester thereof wherein Y is N and R5 is optionally substituted C(═O)—C₅₋₁₀ aryl or C(═O)—C₅₋₁₀ heteroaryl.

3. A compound as claimed in claim 1 or a pharmaceutically acceptable salt or an in vivo hydrolysable ester thereof wherein Y is N and R5 is optionally substituted SO₂—C₅₋₁₀ aryl or SO₂—C₅₋₁₀ heteroaryl.

4. A compound as claimed in claim 1 or a pharmaceutically acceptable salt or an in vivo hydrolysable ester thereof wherein Y is O and R5 is optionally substituted C₆₋₁₀ aryl-C₁₋₄ alkyl- or C₆₋₁₀ heteroaryl-C₁₋₄ alkyl-.

5. A compound as claimed in claim 1 or a pharmaceutically acceptable salt or an in vivo hydrolysable ester thereof wherein R2 is C₁₋₄ alkyl.

6. (canceled)

7. A pharmaceutical composition comprising a compound of formula (I) as claimed in any one of claims 1-5, or a pharmaceutically acceptable salt, or an in vivo hydrolysable ester thereof, in combination with a pharmaceutically acceptable diluent or carrier

8. A method for the treatment of Mycobacterium tuberculosis which comprises administering to a human or animal an effective amount of a compound of formula (I) as claimed in any one of claims 1-5, or a pharmaceutically acceptable salt, or an in vivo hydrolysable ester thereof.

9. A process for the preparation of a compound of the formula (I) as claimed in claim 1, or a pharmaceutically acceptable salt or an in vivo hydrolysable ester thereof which process comprises:
where Y is N,
by reacting a compound of formula (II) 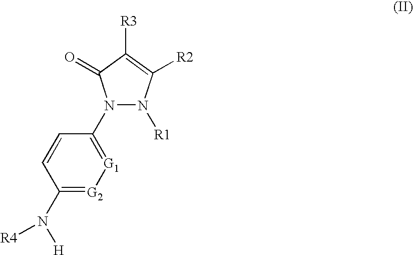
wherein R¹, R², R³, R⁴, G₁ and G₂ are as defined in relation to formula (I), with a compound of formula (III)
R5-SO₂—Z (III)
wherein R⁵ is as defined in relation to formula (I), and
wherein Z is a leaving group, under appropriate reaction conditions; or
(ii) where Y is C═O, by reacting a compound of formula II as defined above, with a compound of formula (IV)
R⁵—CO—Z (IV)
wherein R⁵ is as defined in relation to formula (I), and
wherein Z is a leaving group, under appropriate reaction conditions; or
(iii) Y is O, by reacting a compound of formula (V) 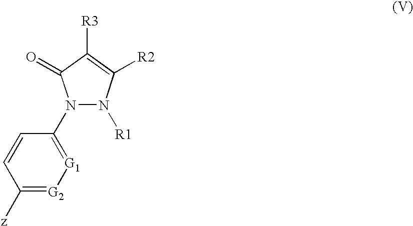
wherein R¹, R², R³, G₁ and G₂ are as defined in relation to formula (I),
wherein Z is a leaving group, with a compound of the formula (VI)
R⁵—OH (VI)
wherein R⁵ is as defined in relation to formula (I), under appropriate reaction conditions;
and thereafter if desired or necessary converting any substituent group to another substituent group as defined.
